(19) United States
(12) Patent Application Publication
(10) Pub. No.: US 2006/0135770 A1
(43) Pub. Date: Jun. 22, 2006

(54) PARP inhibitors

(75) Inventors: Muhammad Hashim Javaid, Cambridge (GB); Graeme Cameron Murray Smith, Cambridge (GB); Niall Morrison Barr Martin, Cambridge (GB); Sylvie Gomez, Cambridge (GB); Vincent Junior Ming Lai Loh, Cambridge (GB); Xiao-Ling Fan Cockcroft, Cambridge (GB); Keith Allan Menear, Cambridge (GB)

Correspondence Address: MICHAEL BEST & FRIEDRICH, LLP, ONE SOUTH PINCKNEY STREET, P O BOX 1806, MADISON, WI, 53701, US

(73) Assignee: Kudos Pharmaceuticals Ltd., Cambridge (GB)

(21) Appl. No.: 11/315,528

(22) Filed: Dec. 22, 2005

Related U.S. Application Data
(60) Provisional application No. 60/638,912, filed on Dec. 23, 2004.
(60) Provisional application No. 60/695,306, filed on Jun. 30, 2005.

(30) Foreign Application Priority Data
Dec. 22, 2004 (GB) 0428111.9

(51) Int. Cl. C07D 403/14

(52) U.S. Cl. 544/295; 544/333

(57) ABSTRACT

A compound of the formula (I): 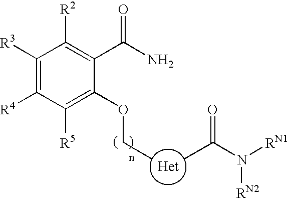
and isomers, salts, solvates, chemically protected forms, and prodrugs thereof, wherein: R², R³, R⁴ and R⁵ are independently selected from the group consisting of H, C₁₋₇ alkoxy, amino, halo or hydroxy; n is 1 or 2; R^{N1} and R^{N2} are independently selected from H and R, where R is optionally substituted C₁₋₁₀ alkyl, C₃₋₂₀ heterocyclyl and C₅₋₂₀ aryl; or R^{N1} and R^{N2}, together with the nitrogen atom to which they are attached form an optionally substituted 5-7 membered, nitrogen containing, heterocylic ring; Het is selected from: 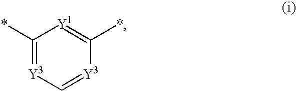
where Y¹ and Y³ are independently selected from CH and N, Y² is selected from CX and N and X is H, Cl or F; and 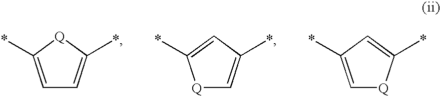 where Q is O or S.

### CROSS-REFERENCE TO RELATED APPLICATIONS

This application claims the priority benefits of U.S. Provisional Patent Application Ser. No. 60/638,912, filed on Dec. 23, 2004 and U.S. Provisional Patent Application Ser. No. 60/695,306, filed Jun. 30, 2005, and claims foreign priority benefits of GB 0428111.9, filed on Dec. 22, 2004, which are herein incorporated by reference.

The present invention relates to succinimide derivatives, and their use as pharmaceuticals. In particular, the present invention relates to the use of these compounds to inhibit the activity of the enzyme poly (ADP-ribose)polymerase, also known as poly(ADP-ribose)synthase and poly ADP-ribosyltransferase, and commonly referred to as PARP.

The mammalian enzyme PARP (a 113-kDa multidomain protein) has been implicated in the signalling of DNA damage through its ability to recognize and rapidly bind to DNA single or double strand breaks (D'Amours, et al., Biochem. J., 342, 249-268 (1999)).

Several observations have led to the conclusion that PARP participates in a variety of DNA-related functions including gene amplification, cell division, differentiation, apoptosis, DNA base excision repair and also effects on telomere length and chromosome stability (d'Adda di Fagagna, et al., Nature Gen., 23(1), 76-80 (1999)).

Studies on the mechanism by which PARP modulates DNA repair and other processes has identified its importance in the formation of poly (ADP-ribose) chains within the cellular nucleus (Althaus, F. R. and Richter, C., ADP-Ribosylation of Proteins: Enzymology and Biological Significance, Springer-Verlag, Berlin (1987)). The DNA-bound, activated PARP utilizes NAD to synthesize poly (ADP-ribose) on a variety of nuclear target proteins, including topoisomerase, histones and PARP itself (Rhun, et al., Biochem. Biophys. Res. Commun., 245,1-10 (1998)).

Poly (ADP-ribosyl)ation has also been associated with malignant transformation. For example, PARP activity is higher in the isolated nuclei of SV40-transformed fibroblasts, while both leukemic cells and colon cancer cells show higher enzyme activity than the equivalent normal leukocytes and colon mucosa (Miwa, et al., Arch. Biochem. Biophys., 181, 313-321 (1977); Burzio, etal., Proc. Soc. Exp. Bioi. Med., 149, 933-938 (1975); and Hirai, et al., Cancer Res., 43, 3441-3446 (1983)).

A number of low-molecular-weight inhibitors of PARP have been used to elucidate the functional role of poly (ADP-ribosyl)ation in DNA repair. In cells treated with alkylating agents, the inhibition of PARP leads to a marked increase in DNA-strand breakage and cell killing (Durkacz, et al., Nature, 283, 593-596 (1980); Berger, N. A., Radiation Research, 101, 4-14 (1985)).

Subsequently, such inhibitors have been shown to enhance the effects of radiation response by suppressing the repair of potentially lethal damage (Ben-Hur, et al., British Joumal of Cancer, 49 (Suppl. VI), 34-42 (1984); Schlicker, et al., Int J. Radiat. Bioi., 75, 91-100 (1999)). PARP inhibitors have been reported to be effective in radio sensitising hypoxic tumour cells (U.S. Pat. No. 5,032,617; U.S. Pat. No. 5,215,738 and U.S. Pat. No. 5,041,653).

Furthermore, PARP knockout (PARP −/−) animals exhibit genomic instability in response to alkylating agents and γ-irradiation (Wang, et al., Genes Dev., 9, 509-520 (1995); Menissier de Murcia, et al., Proc. Natl. Acad. Sci. USA, 94, 7303-7307 (1997)).

A role for PARP has also been demonstrated in certain vascular diseases, septic shock, ischaemic injury and neurotoxicity (Cantoni, et al., Biochim. Biophys. Acta, 1014, 1-7 (1989); Szabo, et al., J. Clin. Invest., 100, 723-735 (1997)). Oxygen radical DNA damage that leads to strand breaks in DNA, which are subsequently recognised by PARP, is a major contributing factor to such disease states as shown by PARP inhibitor studies (Cosi, et al., J. Neurosci. Res., 39, 38-46 (1994); Said, et al., Proc. Natl. Acad. Sci. U.S.A., 93, 4688-4692 (1996)). More recently, PARP has been demonstrated to play a role in the pathogenesis of haemorrhagic shock (Liaudet, et al., Proc. Natl. Acad. Sci. U.S.A., 97(3), 10203-10208 (2000)).

It has also been demonstrated that efficient retroviral infection of mammalian cells is blocked by the inhibition of PARP activity. Such inhibition of recombinant retroviral vector infections was shown to occur in various different cell types (Gaken, et al., J. Virology, 70(6), 3992-4000 (1996)). Inhibitors of PARP have thus been developed for the use in anti-viral therapies and in cancer treatment (WO 91/18591).

Moreover, PARP inhibition has been speculated to delay the onset of aging -characteristics in human fibroblasts (Rattan and Clark, Biochem. Biophys. Res. Comm., 201(2), 665-672 (1994)). This may be related to the role that PARP plays in controlling telomere function (d'Adda di Fagagna, et al., Nature Gen., 23(1), 76-80 (1999)).

PARP inhibitors are also thought to be relevant to the treatment of inflammatory bowel disease (Szabo C., Role of Poly(ADP-Ribose) Polymerase Activation in the Pathogenesis of Shock and Inflammation, In PARP as a Therapeutic Target; Ed J. Zhang, 2002 by CRC Press; 169-204), ulcerative colitis (Zingarelli, B, et al., Immunology, 113(4), 509-517 (2004)) and Crohn's disease (Jijon, H.B., et al., Am. J. Physiol. Gastrointest. Liver Physiol., 279, G641-G651 (2000).

Some of the present inventors have previously described (WO 02/36576) a class of 1(2H)-phthalazinone compounds which act as PARP inhibitors. The compounds have the general formula: 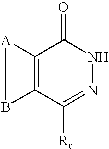
where A and B together represent an optionally substituted, fused aromatic ring and where R_{c} is represented by -L-R_{L}. A large number of examples are of the formula: 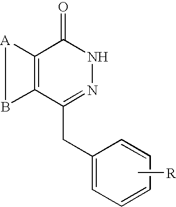
where R represent one or more optional substituents.

The present inventors have now discovered a further class of compounds that inhibit the activity of PARP.

Accordingly, the first aspect of the present invention provides a compound of the formula (I): 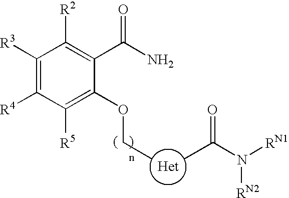
and isomers, salts, solvates, chemically protected forms, and prodrugs thereof, wherein:

R², R³, R⁴ and R⁵ are independently selected from the group consisting of H, C₁₋₇ alkoxy, amino, halo or hydroxy;

n is 1 or 2;

R^{N1} and R^{N2} are independently selected from H and R, where R is optionally substituted C₁₋₁₀ alkyl, C₃₋₂₀ heterocyclyl and C₅₋₂₀ aryl;

or R^{N1} and R^{N2}, together with the nitrogen atom to which they are attached form an optionally substituted 5-7 membered, nitrogen containing, heterocylic ring;

Het is selected from: 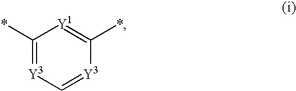
where Y¹ and Y³ are independently selected from CH and N, Y² is selected from CX and N and X is H, Cl or F; and 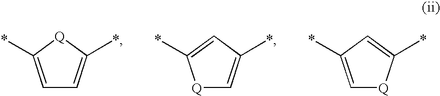
where Q is O or S.

The possibilities for Het are:

Formula

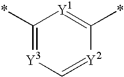

| Y¹ | Y² | Y³ | Group |
|---|---|---|---|
| N | CH | CH | 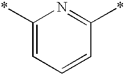 |
| N | CF | CH | 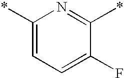 |
| CH | CH | N | 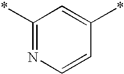 |
| CH | CF | N | 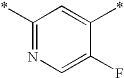 |
| CH | N | CH | 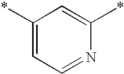 |
| N | CH | N | 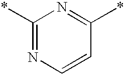 |
| N | CF | N | 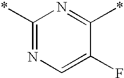 |
| N | N | CH | 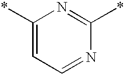 |
| CH | N | N | 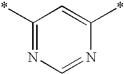 |

| | | |
|---|---|---|
| 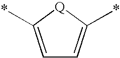 | 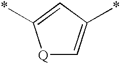 | 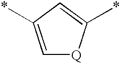 |

Q

| | |
|---|---|
| O | 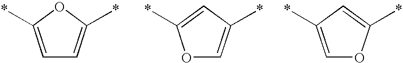 |
| S | 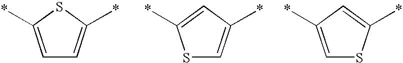 |

A second aspect of the present invention provides a pharmaceutical composition comprising a compound of the first aspect and a pharmaceutically acceptable carrier or diluent.

A third aspect of the present invention provides a compound of the first aspect for use in a method of treatment of the human or animal body.

A fourth aspect of the present invention provides the use of a compound as defined in the first aspect of the invention in the preparation of a medicament for:

(a) inhibiting the activity of PARP (PARP-1 and/or PARP-2);

(b) the treatment of: vascular disease; septic shock; ischaemic injury, both cerebral and cardiovascular; reperfusion injury, both cerebral and cardiovascular; neurotoxicity, including acute and chronic treatments for stroke and Parkinsons disease; haemorraghic shock; inflammatory diseases, such as arthritis, inflammatory bowel disease, ulcerative colitis and Crohn's disease; multiple sclerosis; secondary effects of diabetes; as well as the acute treatment of cytoxicity following cardiovascular surgery or diseases ameliorated by the inhibition of the activity of PARP;

(c) use as an adjunct in cancer therapy or for potentiating tumour cells for treatment with ionizing radiation or chemotherapeutic agents.

In particular, compounds as defined in the first aspect of the invention can be used in anti-cancer combination therapies (or as adjuncts) along with alkylating agents, such as methyl methanesulfonate (MMS), temozolomide and dacarbazine (DTIC), also with topoisomerase-1 inhibitors like Topotecan, Irinotecan, Rubitecan, Exatecan, Lurtotecan, Gimetecan, Diflomotecan (homocamptothecins); as well as 7-substituted non-silatecans; the 7-silyl camptothecins, BNP 1350; and non-camptothecin topoisomerase-I inhibitors such as indolocarbazoles also dual topoisomerase-I and II inhibitors like the benzophenazines, XR 11576/MLN 576 and benzopyridoindoles. Such combinations could be given, for example, as intravenous preparations or by oral administration as dependent on the preferred method of administration for the particular agent.

Another further aspect of the invention provides for the use of a compound as defined in the first aspect of the invention in the preparation of a medicament for use as an adjunct in cancer therapy or for potentiating tumour cells for treatment with ionizing radiation or chemotherapeutic agents.

Other further aspects of the invention provide for the treatment of disease ameliorated by the inhibition of PARP, comprising administering to a subject in need of treatment a therapeutically-effective amount of a compound as defined in the first aspect, preferably in the form of a pharmaceutical composition and the treatment of cancer, comprising administering to a subject in need of treatment a therapeutically-effective amount of a compound as defined in the first aspect in combination, preferably in the form of a pharmaceutical composition, simultaneously or sequentially with ionizing radiation or chemotherapeutic agents.

In further aspects of the present invention, the compounds may be used in the preparation of a medicament for the treatment of cancer which is deficient in Homologous Recombination (HR) dependent DNA double strand break (DSB) repair activity, or in the treatment of a patient with a cancer which is deficient in HR dependent DNA DSB repair activity, comprising administering to said patient a therapeutically-effective amount of the compound.

The HR dependent DNA DSB repair pathway repairs double-strand breaks (DSBs) in DNA via homologous mechanisms to reform a continuous DNA helix (K. K. Khanna and S. P. Jackson, Nat. Genet. 27(3): 247-254 (2001)). The components of the HR dependent DNA DSB repair pathway include, but are not limited to, ATM (NM_{—}000051), RAD51 (NM_{—}002875), RAD51L1 (NM_{—}002877), RAD51C (NM_{—}002876), RAD51L3 (NM_{—}002878), DMC1 (NM_{—}007068), XRCC2 (NM_{—}005431), XRCC3 (NM_{—}005432), RAD52 (NM_{—}002879), RAD54L (NM_{—}003579), RAD54B (NM_{—}012415), BRCA1 (NM_{—}007295), BRCA2 (NM_{—}000059), RAD50 (NM_{—}005732), MRE1 1A (NM_{—}005590) and NBS1 (NM_{—}002485). Other proteins involved in the HR dependent DNA DSB repair pathway include regulatory factors such as EMSY (Hughes-Davies, et al., Cell, 115, pp523-535). HR components are also described in Wood, et al., Science, 291,1284-1289 (2001).

A cancer which is deficient in HR dependent DNA DSB repair may comprise or consist of one or more cancer cells which have a reduced or abrogated ability to repair DNA DSBs through that pathway, relative to normal cells i.e. the activity of the HR dependent DNA DSB repair pathway may be reduced or abolished in the one or more cancer cells.

The activity of one or more components of the HR dependent DNA DSB repair pathway may be abolished in the one or more cancer cells of an individual having a cancer which is deficient in HR dependent DNA DSB repair. Components of the HR dependent DNA DSB repair pathway are well characterised in the art (see for example, Wood, et al., Science, 291,1284-1289 (2001)) and include the components listed above.

In some preferred embodiments, the cancer cells may have a BRCA1 and/or a BRCA2 deficient phenotype i.e. BRCA1 and/or BRCA2 activity is reduced or abolished in the cancer cells. Cancer cells with this phenotype may be deficient in BRCA1 and/or BRCA2, i.e. expression and/or activity of BRCAI and/or BRCA2 may be reduced or abolished in the cancer cells, for example by means of mutation or polymorphism in the encoding nucleic acid, or by means of amplification, mutation or polymorphism in a gene encoding a regulatory factor, for example the EMSY gene which encodes a BRCA2 regulatory factor (Hughes-Davies, et al., Cell, 115, 523-535) or by an epigenetic mechanism such as gene promoter methylation.

BRCA1 and BRCA2 are known tumour suppressors whose wild-type alleles are frequently lost in tumours of heterozygous carriers (Jasin M., Oncogene, 21(58), 8981-93 (2002); Tutt, et al., Trends Mol Med., 8(12), 571-6, (2002)). The association of BRCA1 and/or BRCA2 mutations with breast cancer is well-characterised in the art (Radice, P. J., Exp Clin Cancer Res., 21(3 Suppl), 9-12 (2002)). Amplification of the EMSY gene, which encodes a BRCA2 binding factor, is also known to be associated with breast and ovarian cancer.

Carriers of mutations in BRCA1 and/or BRCA2 are also at elevated risk of cancer of the ovary, prostate and pancreas.

In some preferred embodiments, the individual is heterozygous for one or more variations, such as mutations and polymorphisms, in BRCA1 and/or BRCA2 or a regulator thereof. The detection of variation in BRCA1 and BRCA2 is well-known in the art and is described, for example in EP 699 754, EP 705 903, Neuhausen, S. L. and Ostrander, E. A., Genet. Test, 1, 75-83 (1992); Janatova M., et al., Neoplasma, 50(4), 246-50 (2003). Determination of amplification of the BRCA2 binding factor EMSY is described in Hughes-Davies, et al., Cell, 115, 523-535).

Mutations and polymorphisms associated with cancer may be detected at the nucleic acid level by detecting the presence of a variant nucleic acid sequence or at the protein level by detecting the presence of a variant (i.e. a mutant or allelic variant) polypeptide.

The above is described in co-pending PCT/GB2004/005025, and a US application, both filed on 30 Nov. 2004 and entitled “DNA damage repair inhibitors for the treatment of cancer”, which are herein incorporated by reference.

### DEFINITIONS

5-7 membered, nitrogen containing, heterocylic ring: This ring must contain at least one nitrogen atom, and may contain further hetero atoms, i.e. O, S, N.

Examples of five to seven membered nitrogen containing heterocyclic rings are set out below, where Cf indicates the number of ring atoms as n.

N₁: pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

N₁O₁S₁: oxathiazine (C₆).

Alkyl: The term “alkyl” as used herein, pertains to a monovalent moiety obtained by removing a hydrogen atom from a carbon atom of a hydrocarbon compound having from 1 to 20 carbon atoms (unless otherwise specified), which may be aliphatic or alicyclic, and which may be saturated or unsaturated (e.g. partially unsaturated, fully unsaturated). Thus, the term “alkyl” includes the sub-classes alkenyl, alkynyl, cycloalkyl, cycloalkyenyl, cylcoalkynyl, etc., discussed below.

In the context of alkyl groups, the prefixes (e.g. C₁₋₄, C₁₋₇, C₁₋₂₀, C₂₋₇, C₃₋₇, etc.) denote the number of carbon atoms, or range of number of carbon atoms. For example, the term “C₁₋₄ alkyl”, as used herein, pertains to an alkyl group having from 1 to 4 carbon atoms. Examples of groups of alkyl groups include C₁₋₄ alkyl (“lower alkyl”), C₁₋₇ alkyl, C₁₋₁₀ alkyl and C₁₋₂₀ alkyl. Note that the first prefix may vary according to other limitations; for example, for unsaturated alkyl groups, the first prefix must be at least 2; for cyclic alkyl groups, the first prefix must be at least 3; etc.

Examples of (unsubstituted) saturated alkyl groups include, but are not limited to, methyl (C₁), ethyl (C₂), propyl (C₃), butyl (C₄), pentyl (C₅), hexyl (C₆), heptyl (C₇), octyl (C₈), nonyl (C₉), decyl (C₁₀), undecyl (C₁₁), dodecyl (C₁₂), tridecyl (C₁₃), tetradecyl (C₁₄), pentadecyl (C₁₅), and eicodecyl (C₂₀).

Examples of (unsubstituted) saturated linear alkyl groups include, but are not limited to, methyl (C₁), ethyl (C₂), n-propyl (C₃), n-butyl (C₄), n-pentyl (amyl) (C₅), n-hexyl (C₆), and n-heptyl (C₇).

Examples of (unsubstituted) saturated branched alkyl groups include iso-propyl (C₃), iso-butyl (C₄), sec-butyl (C₄), tert-butyl (C₄), iso-pentyl (C₅), and neo-pentyl (C₅).

Alkenyl: The term “alkenyl”, as used herein, pertains to an alkyl group having one or more carbon-carbon double bonds. Examples of groups of alkenyl groups include C₂₋₄ alkenyl, C₂₋₇ alkenyl, C₂₋₂₀ alkenyl.

Examples of (unsubstituted) unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

Alkynyl: The term “alkynyl”, as used herein, pertains to an alkyl group having one or more carbon-carbon triple bonds. Examples of groups of alkynyl groups include C₂₋₄ alkynyl, C₂₋₇ alkynyl, C₂₋₂₀ alkynyl.

Examples of (unsubstituted) unsaturated alkynyl groups include, but are not limited to, ethynyl (ethinyl, —C≡CH) and 2-propynyl (propargyl, —CH₂—C≡CH).

Cycloalkyl: The term “cycloalkyl”, as used herein, pertains to an alkyl group which is also a cyclyl group; that is, a monovalent moiety obtained by removing a hydrogen atom from an alicyclic ring atom of a carbocyclic ring of a carbocyclic compound, which carbocyclic ring may be saturated or unsaturated (e.g. partially unsaturated, fully unsaturated), which moiety has from 3 to 20 carbon atoms (unless otherwise specified), including from 3 to 20 ring atoms. Thus, the term “cycloalkyl” includes the sub-classes cycloalkenyl and cycloalkynyl. Preferably, each ring has from 3 to 7 ring atoms. Examples of groups of cycloalkyl groups include C₃₋₂₀ cycloalkyl, C₃₋₁₅ cycloalkyl, C₃₋₁₀ cycloalkyl, C₃₋₇ cycloalkyl.

Examples of cycloalkyl groups include, but are not limited to, those derived from:

saturated monocyclic hydrocarbon compounds:
cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇), methylcyclohexane (C₇), dimethylcyclohexane (C₈), menthane (C₁₀);

unsaturated monocyclic hydrocarbon compounds:
cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇), methylcyclohexene (C₇), dimethylcyclohexene (C₈);

saturated polycyclic hydrocarbon compounds:
thujane (C₁₀), carane (C₁₀), pinane (C₁₀), bornane (C₁₀), norcarane (C₇), norpinane (C₇), norbornane (C₇), adamantane (C₁₀), decalin (decahydronaphthalene) (C₁₀);

unsaturated polycyclic hydrocarbon compounds:
camphene (C₁₀), limonene (C₁₀), pinene (C₁₀);

polycyclic hydrocarbon compounds having an aromatic ring:
indene (C₉), indane (e.g., 2,3-dihydro-1H-indene) (C₉), tetraline (1,2,3,4-tetrahydronaphthalene) (C₁₀), acenaphthene (C₁₂), fluorene (C₁₃), phenalene (C₁₃), acephenanthrene (C₁₅), aceanthrene (C,₆), cholanthrene (C₂₀).

Heterocyclyl: The term “heterocyclyl”, as used herein, pertains to a monovalent moiety obtained by removing a hydrogen atom from a ring atom of a heterocyclic compound, which moiety has from 3 to 20 ring atoms (unless otherwise specified), of which from 1 to 10 are ring heteroatoms. Preferably, each ring has from 3 to 7 ring atoms, of which from 1 to 4 are ring heteroatoms.

In this context, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms. Examples of groups of heterocyclyl groups include C₃₋₂₀ heterocyclyl, C₅₋₂₀ heterocyclyl, C₃₋₁₅ heterocyclyl, C₅₋₁₅ heterocyclyl, C₃₋₁₂ heterocyclyl, C₅₋₁₂ heterocyclyl, C₃₋₁₀ heterocyclyl, C₅₋₁₀ heterocyclyl, C₃₋₇ heterocyclyl, C₅₋₇ heterocyclyl, and C₅₋₆ heterocyclyl.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and, N₁O₁S₁: oxathiazine (C₆).

Examples of substituted (non-aromatic) monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

Spiro-C₃₋₇ cycloalkyl or heterocyclyl: The term “spiro C₃₋₇ cycloalkyl or heterocyclyl” as used herein, refers to a C₃₋₇ cycloalkyl or C₃₋₇ heterocyclyl ring joined to another ring by a single atom common to both rings.

C₅₋₂₀ aryl: The term “C₅₋₂₀ aryl” as used herein, pertains to a monovalent moiety obtained by removing a hydrogen atom from an aromatic ring atom of a C₅₋₂₀ aromatic compound, said compound having one ring, or two or more rings (e.g., fused), and having from 5 to 20 ring atoms, and wherein at least one of said ring(s) is an aromatic ring. Preferably, each ring has from 5 to 7 ring atoms.

The ring atoms may be all carbon atoms, as in “carboaryl groups” in which case the group may conveniently be referred to as a “C₅₋₂₀ carboaryl” group.

Examples of C₅₋₂₀ aryl groups which do not have ring heteroatoms (i.e. C₅₋₂₀ carboaryl groups) include, but are not limited to, those derived from benzene (i.e. phenyl) (C₆), naphthalene (C₁₀), anthracene (C₁₄), phenanthrene (C₁₄), and pyrene (C₁₆).

Alternatively, the ring atoms may include one or more heteroatoms, including but not limited to oxygen, nitrogen, and sulfur, as in “heteroaryl groups”. In this case, the group may conveniently be referred to as a “C₅₋₂₀ heteroaryl” group, wherein “C₅₋₂₀” denotes ring atoms, whether carbon atoms or heteroatoms. Preferably, each ring has from 5 to 7 ring atoms, of which from 0 to 4 are ring heteroatoms.

Examples of C₅₋₂₀ heteroaryl groups include, but are not limited to, C₅ heteroaryl groups derived from furan (oxole), thiophene (thiole), pyrrole (azole), imidazole (1,3-diazole), pyrazole (1,2-diazole), triazole, oxazole, isoxazole, thiazole, isothiazole, oxadiazole, tetrazole and oxatriazole; and C₆ heteroaryl groups derived from isoxazine, pyridine (azine), pyridazine (1,2-diazine), pyrimidine (1,3-diazine; e.g., cytosine, thymine, uracil), pyrazine (1,4-diazine) and triazine.

The heteroaryl group may be bonded via a carbon or hetero ring atom.

Examples of C₅₋₂₀ heteroaryl groups which comprise fused rings, include, but are not limited to, C₉ heteroaryl groups derived from benzofuran, isobenzofuran, benzothiophene, indole, isoindole; C₁₀ heteroaryl groups derived from quinoline, isoquinoline, benzodiazine, pyridopyridine; C₁₄ heteroaryl groups derived from acridine and xanthene.

The above alkyl, heterocyclyl, and aryl groups, whether alone or part of another substituent, may themselves optionally be substituted with one or more groups selected from themselves and the additional substituents listed below.

Halo: —F, —Cl, —Br, and —I.

Hydroxy: —OH.

Ether: —OR, wherein R is an ether substituent, for example, a C₁₋₇ alkyl group (also referred to as a C₁₋₇ alkoxy group), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇ alkyl group.

Nitro: —NO₂.

Cyano (nitrile, carbonitrile): —CN.

Acyl (keto): —C(═O)R, wherein R is an acyl substituent, for example, H, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (butyryl), and —C(═O)Ph (benzoyl, phenone).

Carboxy (carboxylic acid): —COOH.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Amido (carbamoyl, carbamyl, aminocarbonyl, carboxamide): —C(═O)NR¹R², wherein R¹ and R² are independently amino substituents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinylcarbonyl.

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Examples of amino groups include, but are not limited to, —NH₂, —NHCH₃, —NHCH(CH₃)₂, —N(CH₃)₂, —N(CH₂CH₃)₂, and —NHPh. Examples of cyclic amino groups include, but are not limited to, aziridinyl, azetidinyl, pyrrolidinyl, piperidino, piperazinyl, perhydrodiazepinyl, morpholino, and thiomorpholino. The cylic amino groups may be substituted on their ring by any of the substituents defined here, for example carboxy, carboxylate and amido.

Acylamido (acylamino): —NR¹C(═O)R², wherein R¹ is an amide substituent, for example, hydrogen, a C₁₋₇ alkyl group, a C₃₋₂₀ heterocyclyl group, or a C₅₋₂₀ aryl group, preferably H or a C₁₋₇ alkyl group, most preferably H, and R² is an acyl substituent, for example, a C₁₋₇ alkyl group, a C₃₋₂₀ heterocyclyl group, or a C₅₋₂₀ aryl group, preferably a C₁₋₇ alkyl group. Examples of acylamide groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl:

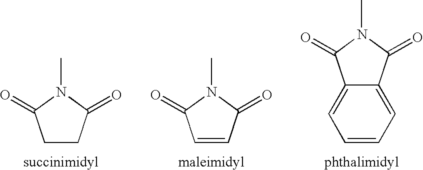

Ureido: —N(R¹)CONR²R³ wherein R² and R³ are independently amino substituents, as defined for amino groups, and R1 is a ureido substituent, for example, hydrogen, a C₁₋₇alkyl group, a C₃₋₂₀heterocyclyl group, or a C₅₋₂₀aryl group, preferably hydrogen or a C₁₋₇alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, —NMeCONEt₂ and —NHC(═O)NHPh.

Acyloxy (reverse ester): —OC(═O)R, wherein R is an acyloxy substituent, for example, a C₁₋₇ alkyl group,, a C₃₋₂₀ heterocyclyl group, or a C₅₋₂₀ aryl group, preferably a C₁₋₇ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, —OC(═O)C₆H₄F, and —OC(═O)CH₂Ph.

Thiol: —SH.

Thioether (sulfide): —SR, wherein R is a thioether substituent, for example, a C₁₋₇ alkyl group (also referred to as a C₁₋₇ alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Sulfoxide (sulfinyl): —S(═O)R, wherein R is a sulfoxide substituent, for example, a C₁₋₇ alkyl group, a C₃₋₂₀ heterocyclyl group, or a C₅₋₂₀ aryl group, preferably a C₁₋₇ alkyl group. Examples of sulfoxide groups include, but are not limited to, —S(═O)CH₃ and —S(═O)CH₂CH₃.

Sulfonyl (sulfone): —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group. Examples of sulfone groups include, but are not limited to, —S(═O)₂CH₃ (methanesulfonyl, mesyl), —S(═O)₂CF₃, —S(═O)₂CH₂CH₃, and 4-methylphenylsulfonyl (tosyl).

Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Sulfonamino: —NR¹S(═O)₂R, wherein R¹ is an amino substituent, as defined for amino groups, and R is a sulfonamino substituent, for example, a C₁₋₇alkyl group, a C₃₋₂₀heterocyclyl group, or a C₅₋₂₀aryl group, preferably a C₁₋₇alkyl group. Examples of sulfonamino groups include, but are not limited to, —NHS(═O)₂CH₃, —NHS(═O)₂Ph and —N(CH₃)S(═O)₂C₆H₅.

As mentioned above, the groups that form the above listed substituent groups, e.g. C₁₋₇ alkyl, C₃₋₂₀ heterocyclyl and C₅₋₂₀ aryl, may themselves be substituted. Thus, the above definitions cover substituent groups which are substituted.

### FURTHER PREFERENCES

The following preferences can apply to each aspect of the present invention, where applicable.

R², R³, R⁴ and R⁵ are preferably selected from the group consisting of H, C₁₋₇ alkoxy, Cl and F. If one of R², R³, R⁴ and R⁵ is C₁₋₇ alkoxy it is preferably OMe.

R², R³, R⁴ and R⁵ are more preferably selected from the group consisting of H and F R², R⁴ and R⁵ are most preferably H. R³ is most preferably selected from H and F.

In some embodiments, n is preferably 1. In other embodiments, n is preferably 2.

Het is preferably

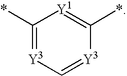

It is preferred that upto two of Y¹, Y² and Y³ are N, and more preferred that one or none of Y¹, Y² and Y³ are N. If one of Y¹, Y² and Y³ are N, it is preferred that this is either Y¹ or Y²

X is preferably selected from H and F, with F being more preferred when n is 1 and H being more preferred when n is 2.

If Het is 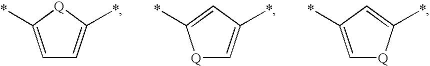
then Q is preferably S. Of these groups, 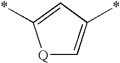
is preferred.

If R^{N1} and R^{N2} are selected from H and R, it is preferred that R^{N1} is H and R^{N2} is R. R is preferably optionally substituted C₁₋₇ alkyl or C₃₋₂₀ heterocylyl, with optionally substituted C₁₋₇ alkyl being more preferred. The C₁₋₇ alkyl group is preferably unsubstituted or substituted with a single substituent, which is preferably selected from a C₅₋₂₀ heterocyclic group (e.g. piperidyl, N-methyl pyrrolyl, tetrahydrofuranyl), a C₅₋₂₀ aryl group (e.g. furanyl, phenyl, pyridyl), amino (e.g. dimethyl amino), halo (e.g. Cl, F), hydroxy, ether (e.g. C₁₋₇ alkoxy), thioether (e.g. C₁₋₇ alkylthio). More preferably the single substituent is selected from a C₅₋₂₀ heterocyclic group (e.g. piperidyl, N-methyl pyrrolyl, tetrahydrofuranyl), a C₅₋₂₀ aryl group (e.g. furanyl, phenyl, pyridyl), amino (e.g. dimethyl amino), and ether (e.g. C₁₋₇ alkoxy).

When R^{N1} and R^{N2}, together with the nitrogen atom to which they are attached form a 5 to 7 membered, nitrogen containing heterocyclic ring, they preferably form a group of formula II: 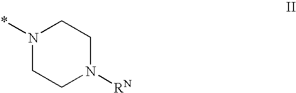
wherein R^{N} is selected from:

(i) -R^{II};

(ii) —C(═O)NHR^{II};

(iii) —C(═S)NHR^{II};

(iv) —S(═O)₂R^{II}; and

(v) —C(═O)R^{II},

where R^{II} is as defined earlier (i.e. optionally substituted C₁₋₁₀ alkyl, C₃₋₂₀ heterocyclyl and C₅₋₂₀ aryl).

Preferably, R^{N} is selected from:

(i) —C(═O)NHR^{II};

(ii) —S(═O)₂R^{II}; and

(iii) —C(═O)R^{II},

where R^{II} is as defined earlier (i.e. optionally substituted C₁₋₂₀ alkyl, C₃₋₂₀ heterocyclyl and C₅₋₂₀ aryl).

In the group of formula II, R^{II} is preferably selected from optionally substituted C₁₋₁₀ alkyl and C₅₋₂₀ aryl.

When R^{II} is C₁₋₁₀ alkyl, it is preferably selected from C₁₋₇ alkyl, for example methyl, ethyl, iso-propyl, n-butyl, tert-butyl and C₃₋₆ cycloalkyl, which may be optionally substituted.

When R^{II} is C₁₋₁₀ alkyl, and in particular linear and branched C₁₋₇ alkyl, it may be optionally substituted by one or more, preferably one, groups selected from, for example: C₅₋₂₀ aryl (e.g. phenyl, methyl phenyl, dimethoxy phenyl), C₂₋₂₀ aryloxy (e.g. phenyloxy), C₃₋₂₀ heterocylyl (e.g. piperidinyl), C₁₋₇ alkoxy (e.g. methoxy, benzyloxy).

When R^{II} is C₅₋₂₀ aryl, it is may be selected from optionally substituted C₅₋₆ aryl (e.g. phenyl, oxazole, isoxazole, pyrazole) and optionally substituted C₈₋₁₀ aryl (e.g. benzyloxadiazole, thianopyrazole).

When R^{II} is C₅₋₂₀ aryl, and in particular C₅₋₆ aryl and C₈₋₁₀ aryl, it may be optionally substituted by one or more groups selected from, for example: halo (e.g. F, Cl), C₁₋₇ alkyl (e.g. Me, CF₃), C₅₋₂₀ aryloxy (e.g. phenyloxy), C₁₋₇ alkoxy (e.g. methoxy, benzyloxy), acylamido (e.g. —NH—C(═O)—Me).

When R^{N1} and R^{N2}, together with the nitrogen atom to which they are attached form a 5 to 7 membered, nitrogen containing heterocyclic ring, they may form a group of formula III: 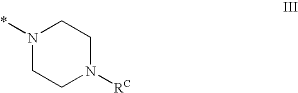
wherein R^{C} is preferably selected from the group consisting of: H; optionally substituted C₁₋₂₀ alkyl; optionally substituted C₅₋₂₀ aryl; optionally substituted C₃₋₂₀ heterocyclyl; optionally substituted acyl, wherein the acyl substituent is preferably selected from C₅₋₂₀ aryl and C₃₋₂₀ heterocylyl (e.g. piperazinyl); optionally substituted amido, wherein the amino groups are preferably selected from H and C₁₋₂₀ alkyl or together with the nitrogen atom, form a C₅₋₂₀ heterocyclic group; and optionally substituted ester groups, wherein the ester substituent is preferably selected from C₁₋₂₀ alkyl groups.

R^{C} is more preferably selected from optionally substituted ester groups, wherein the ester substituent is preferably selected from C₁₋₂₀ alkyl groups.

Particularly preferred compounds include: 53, 71, 72, 74, 79 and 155.

Where appropriate, the above preferences may be taken in combination with each other.

### Includes Other Forms

Included in the above are the well known ionic, salt, solvate, and protected forms of these substituents. For example, a reference to carboxylic acid (—COOH) also includes the anionic (carboxylate) form (—COO—), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms of a hydroxyl group.

### Isomers, Salts, Solvates, Protected Forms, and Prodrugs

Certain compounds may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and reforms; endo- and exo-forms; R-, S-, and meso-forms; D- and L-forms; d- and/-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halichair-forms; and combinations thereof, hereinafter collectively referred to as “isomers” (or “isomeric forms”).

If the compound is in crystalline form, it may exist in a number of different polymorphic forms.

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol, imine/enamine, amide/imino alcohol, amidine/amidine, nitroso/oxime, thioketone/enethiol, N-nitroso/hyroxyazo, and nitro/aci-nitro.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below, as well as its different polymorphic forms.

It may be convenient or desirable to prepare, purify, and/or handle a corresponding salt of the active compound, for example, a pharmaceutically-acceptable salt. Examples of pharmaceutically acceptable salts are discussed in Berge, et al., “Pharmaceutically Acceptable Salts”, J. Pharm. Sci., 66, 1-19 (1977).

For example, if the compound is anionic, or has a functional group which may be anionic (e.g., —COOH may be —COO—), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: acetic, propionic, succinic, gycolic, stearic, palmitic, lactic, malic, pamoic, tartaric, citric, gluconic, ascorbic, maleic, hydroxymaleic, phenylacetic, glutamic, aspartic, benzoic, cinnamic, pyruvic, salicyclic, sulfanilic, 2-acetyoxybenzoic, fumaric, toluenesulfonic, methanesulfonic, ethanesulfonic, ethane disulfonic, oxalic, isethionic, valeric, and gluconic. Examples of suitable polymeric anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form,” as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, “Protective Groups in Organic Synthesis” (T. Green and P. Wuts; 3rd Edition; John Wiley and Sons, 1999).

For example, a hydroxy group may be protected as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc).

For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide or a urethane, for example, as: a methyl amide (—NHCO—CH₃); a benzyloxy amide (—NHCO—OCH₂C₆H₅, —NH—Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl) ethyloxy amide (—NH-Psec); or, in suitable cases, as an N-oxide (>NO□).

For example, a carboxylic acid group may be protected as an ester for example, as: an C₁₋₇ alkyl ester (e.g. a methyl ester; a t-butyl ester); a C₁₋₇ haloalkyl ester (e.g. a C₁₋₇ trihaloalkyl ester); a triC₁₋₇ alkylsilyl-C₁₋₇ alkyl ester; or a C₅₋₂₀ aryl-C₁₋₇ alkyl ester (e.g. a benzyl ester; a nitrobenzyl ester); or as an amide, for example, as a methyl amide.

For example, a thiol group may be protected as a thioether (-SR), for example, as: a benzyl thioether; an acetamidomethyl ether (—S—CH₂NHC(═O)CH₃).

It may be convenient or desirable to prepare, purify, and/or handle the active compound in the form of a prodrug. The term “prodrug”, as used herein, pertains to a compound which, when metabolised (e.g. in vivo), yields the desired active compound. Typically, the prodrug is inactive, or less active than the active compound, but may provide advantageous handling, administration, or metabolic properties.

For example, some prodrugs are esters of the active compound (e.g. a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required. Examples of such metabolically labile esters include those wherein R is C₁₋₂₀alkyl (e.g. -Me, -Et); C₁₋₇aminoalkyl (e.g. aminoethyl; 2-(N,N-diethylamino)ethyl; 2-(4-morpholino)ethyl); and acyloxy-C₁₋₇ alkyl (e.g. acyloxymethyl; acyloxyethyl; e.g. pivaloyloxymethyl; acetoxymethyl; 1-acetoxyethyl; 1-(1-methoxy-1-methyl)ethyl-carbonxyloxyethyl; 1-(benzoyloxy)ethyl; isopropoxy-carbonyloxymethyl; 1-isopropoxy-carbonyloxyethyl; cyclohexyl-carbonyloxymethyl; 1-cyclohexyl-carbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetrahydropyranyloxy)carbonyloxyethyl; (4-tetrahydropyranyl) carbonyloxymethyl; and 1-(4-tetrahydropyranyl) carbonyloxyethyl).

Further suitable prodrug forms include phosphonate and glycolate salts. In particular, hydroxy groups (—OH), can be made into phosphonate prodrugs by reaction with chlorodibenzylphosphite, followed by hydrogenation, to form a phosphonate group —O— P(═O)(OH)₂. Such a group can be cleared by phosphotase enzymes during metabolism to yield the active drug with the hydroxy group.

Also, some prodrugs are activated enzymatically to yield the active compound, or a compound which, upon further chemical reaction, yields the active compound. For example, the prodrug may be a sugar derivative or other glycoside conjugate, or may be an amino acid ester derivative.

### Acronyms

For convenience, many chemical moieties are represented using well known abbreviations, including but not limited to, methyl (Me), ethyl (Et), n-propyl (nPr), iso-propyl (iPr), n-butyl (nBu), tert-butyl (tBu), n-hexyl (nHex), cyclohexyl (cHex), phenyl (Ph), biphenyl (biPh), benzyl (Bn), naphthyl (naph), methoxy (MeO), ethoxy (EtO), benzoyl (Bz), and acetyl (Ac).

For convenience, many chemical compounds are represented using well known abbreviations, including but not limited to, methanol (MeOH), ethanol (EtOH), iso-propanol (i-PrOH), methyl ethyl ketone (MEK), ether or diethyl ether (Et₂O), acetic acid (AcOH), dichloromethane (methylene chloride, DCM), trifluoroacetic acid (TFA), dimethylformamide (DMF), tetrahydrofuran (THF), and dimethylsulfoxide (DMSO).

### Synthesis

Compounds of the present invention are of formula I: 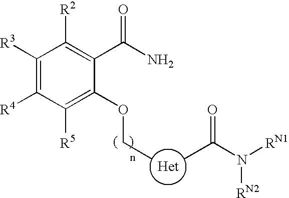
and can be synthesised from a compound of formula 2: 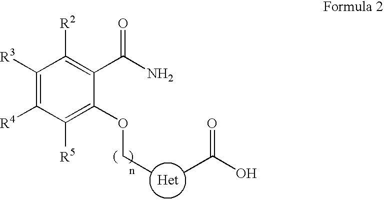
by coupling an amine of formula 3: 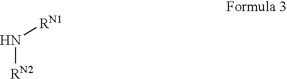
or a precursor or protected form thereof (see below). The coupling may be carried out in the presence of a coupling reagent system, for example 2-(1H-benzotriazol-1-yl)-1,1,3,3-tetramethyluronium tetrafluoroborate, 2-(1H-benzotriazol-1-yl)-1,1,3,3-tetramethyluronium hexafluorophosphate or (dimethylaminopropyl)ethylcarbodiimide hydrochloride/hydroxybenzotriazole, in the presence of a base, for example diisopropylethylamine (Hunig's base), in a solvent, for example dimethylacetamide or dichloromethane, at a temperature in the range of 0° C. to the boiling point of the solvent used.

Alternatively, compounds of the present invention may be synthesised by conversion of a compound of Formula 2 into an activated species, for example an acid chloride or an activated ester such as an N-hydroxysuccinimide ester, using well-known methodologies, and reaction of the activated species with a compound of Formula 3.

Compounds of formula 2 may be obtained by deprotecting compounds of formula 4: 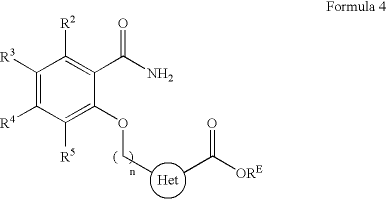
where R^{E} is an optionally substituted, C₁₋₇ alkyl, C₃₋₂₀ heterocyclyl or C₅₋₂₀ aryl group.

Compounds of formula 4 may be synthesised by coupling a compound of formula 5: 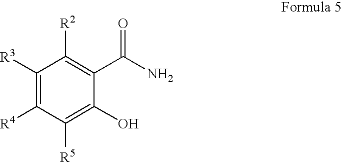
with a compound of formula 6: 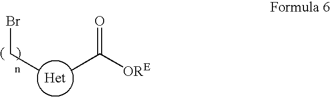
or with a compound of formula 7:

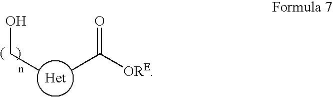

The coupling of compounds of formulae 5 and 6 can be achieved under mildly basic conditions (Williamson reaction), for example, potassium carbonate in acetone.

The coupling of compounds of formulae 5 and 7 can be achieved, using the Mitsunobu reaction (e.g. using diisopropyl azodicarboxylate and triphenylphosphine in acetone).

Compounds of formulae 5, 6 and 7 are either commercially available or readily synthesiable (see examples).

When, in compounds of the present invention, R^{N1} and R^{N2} and the nitrogen atom to which they are attached form a group of formula II: 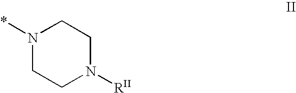
then these compounds can be represented by formula 1a:

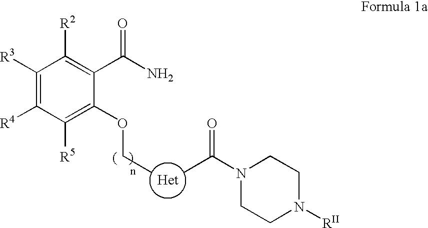

Compounds of formula 1a, wherein R^{II} is H, can be represented by formula 7: 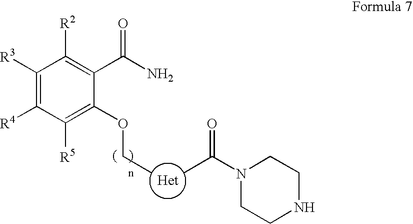
and may be synthesised by deprotection of a protected form of a compound of formula 7, for example a compound of formula 8: 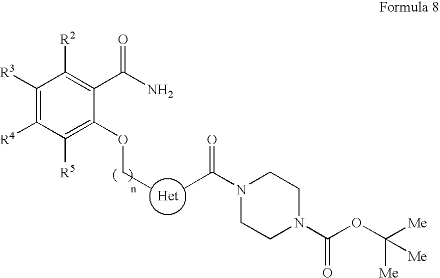
using well known methodologies, for example acid-catalysed cleavage, in the presence of an acid, for example trifluoroacetic acid or hydrochloric acid, in the presence of a solvent, for example dichloromethane or ethanol and/or water, at a temperature in the range of 0° C. to the boiling point of the solvent used.

Compounds of formula 8 may be synthesised from compounds of formula 2 by the previously described methods.

Compounds of formula 1a in which R^{II} is an acyl moiety, can be represented by Formula 9: 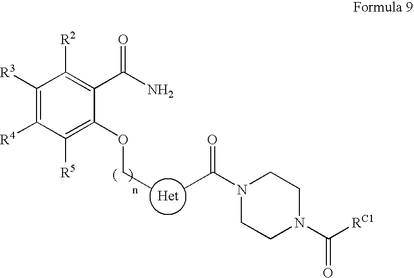
in which R^{C1} is selected from the group consisting of optionally substituted C₁₋₂₀ alkyl, C₅₋₂₀ aryl and C₃₋₂₀ heterocyclyl, and may be synthesised by reaction of a compound of formula 7 with a compound of formula R^{C1}COQ, in which R^{C3} is as previously defined and Q is a suitable leaving group, for example a halogen such as chloro, optionally in the presence of a base, for example pyridine, triethylamine or diisopropylethylamine, optionally in the presence of a solvent, for example dichloromethane, at a temperature in the range of 0° C. to the boiling point of the solvent used.

Compounds of formula 9 may also be synthesised by reaction of a compound of formula 7 with a compound of formula R^{C1}CO₂H, in which R^{C1} is as previously defined, in the presence of a coupling reagent system, for example 2-(1H-benzotriazol-1-yl)-1,1,3,3-tetramethyluronium tetrafluoroborate, 2-(1H-benzotriazol-1-yl)-1,1,3,3-tetramethyluronium hexafluorophosphate or (dimethylaminopropyl)ethylcarbodiimide hydrochloride/hydroxybenzotriazole, in the presence of a base, for example diisopropylethylamine, in a solvent, for example dimethylacetamide or dichloromethane, at a temperature in the range of 0° C. to the boiling point of the solvent used.

Compounds of formula 1a in which R^{II} is an amido or thioamido moiety, can be represented by formula 10: 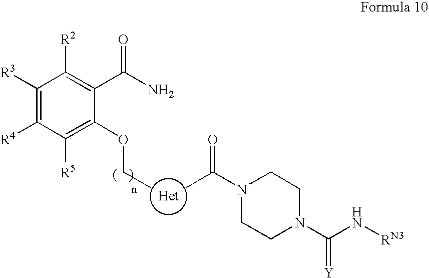
in which Y is O or S, and R^{N3} is selected from the group consisting of optionally substituted C₁₋₂₀ alkyl, C₅₋₂₀ aryl and C₃₋₂₀ heterocyclyl, and may be synthesised by reaction of a compound of formula 7 with a compound of formula R^{N3}NC(═Y), in which R^{N1} are as previously defined, in the presence of a solvent, for example dichloromethane, at a temperature in the range of 0° C. to the boiling point of the solvent used.

Compounds of formula 1a in which R^{II} is a sulfonyl moiety, can be represented by formula 11: 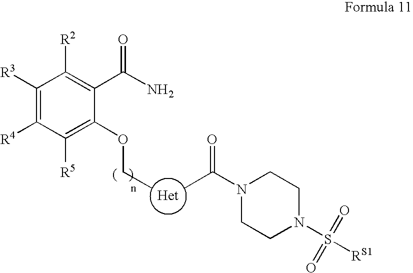
in which R^{S1} is selected from the group consisting of optionally substituted C₁₋₂₀ alkyl, C₅₋₂₀ aryl and C₃₋₂₀ heterocyclyl, and can be synthesised by reaction of a compound of formula 7 with a compound of formula R^{S1}SO₂Cl, in which R^{S1} is as previously defined, optionally in the presence of a base, for example pyridine, triethylamine or diisopropylethylamine, in the presence of a solvent, for example dichloromethane, at a temperature in the range of 0° C. to the boiling point of the solvent used.

Compounds of formula 8: 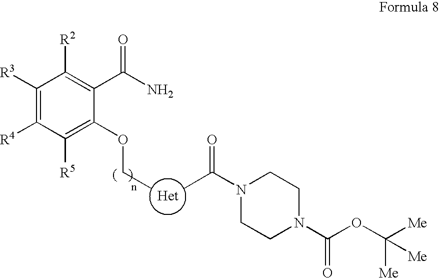
may also be synthesized from compounds of formula 12: 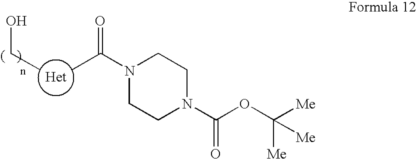
by Mitsunobo coupling with a compound of formula 13:

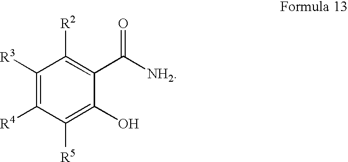

Compounds of formula 12 may be derived from compounds of formula 14: 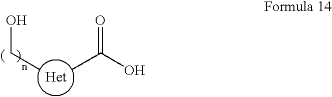
in an analgous way to compounds of formula 8 from compounds of formula 2.

### Use

The present invention provides active compounds, specifically, active in inhibiting the activity of PARP.

The term “active” as used herein, pertains to compounds which are capable of inhibiting PARP activity, and specifically includes both compounds with intrinsic activity (drugs) as well as prodrugs of such compounds, which prodrugs may themselves exhibit little or no intrinsic activity.

One assay which may conveniently be used in order to assess the PARP inhibition offered by a particular compound is described in the examples below.

The present invention further provides a method of inhibiting the activity of PARP in a cell, comprising contacting said cell with an effective amount of an active compound, preferably in the form of a pharmaceutically acceptable composition. Such a method may be practised in vitro or in vivo.

For example, a sample of cells may be grown in vitro and an active compound brought into contact with said cells, and the effect of the compound on those cells observed. As examples of “effect”, the amount of DNA repair effected in a certain time may be determined. Where the active compound is found to exert an influence on the cells, this may be used as a prognostic or diagnostic marker of the efficacy of the compound in methods of treating a patient carrying cells of the same cellular type.

The term “treatment”, as used herein in the context of treating a condition, pertains generally to treatment and therapy, whether of a human or an animal (e.g. in veterinary applications), in which some desired therapeutic effect is achieved, for example, the inhibition of the progress of the condition, and includes a reduction in the rate of progress, a halt in the rate of progress, amelioration of the condition, and cure of the condition. Treatment as a prophylactic measure (i.e. prophylaxis) is also included.

The term “adjunct” as used herein relates to the use of active compounds in conjunction with known therapeutic means. Such means include cytotoxic regimes of drugs and/or ionising radiation as used in the treatment of different cancer types. In particular, the active compounds are known to potentiate the actions of a number of cancer chemotherapy treatments, which include the topoisomerase class of poisons (e.g. topotecan, irinotecan, rubitecan), most of the known alkylating agents (e.g. DTIC, temozolamide) and platinum based drugs (e.g. carboplatin, cisplatin) used in treating cancer.

Active compounds may also be used as cell culture additives to inhibit PARP, for example, in order to sensitize cells to known chemotherapeutic agents or ionising radiation treatments in vitro.

Active compounds may also be used as part of an in vitro assay, for example, in order to determine whether a candidate host is likely to benefit from treatment with the compound in question.

### Administration

The active compound or pharmaceutical composition comprising the active compound may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

The subject may be a eukaryote, an animal, a vertebrate animal, a mammal, a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutang, gibbon), or a human.

### Formulations

While it is possible for the active compound to be administered alone, it is preferable to present it as a pharmaceutical composition (e.g., formulation) comprising at least one active compound, as defined above, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Thus, the present invention further provides pharmaceutical compositions, as defined above, and methods of making a pharmaceutical composition comprising admixing at least one active compound, as defined above, together with one or more pharmaceutically acceptable carriers, excipients, buffers, adjuvants, stabilisers, or other materials, as described herein.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, diluents, excipients, etc. can be found in standard pharmaceutical texts. See, for example, “Handbook of Pharmaceutical Additives”, 2nd Edition (eds. M. Ash and I. Ash), 2001 (Synapse Information Resources, Inc., Endicott, N.Y., USA), “Remington's Pharmaceutical Sciences”, 20th edition, pub. Lippincott, Williams & Wilkins, 2000; and “Handbook of Pharmaceutical Excipients”, 2nd edition, 1994.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, losenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g., by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g. compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g., sodium lauryl sulfate); and preservatives (e.g., methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include losenges comprising the active compound in a flavored basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebuliser, include aqueous or oily solutions of the active compound.

Formulations suitable for administration by inhalation include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g., by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

### Dosage

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 μg to about 250 mg per kilogram body weight of the subject per day. Where the active compound is a salt, an ester, prodrug, or the like, the amount administered is calculated on the basis of the parent compound and so the actual weight to be used is increased proportionately.

### EXAMPLES

General Experimental Methods

Preparative HPLC

Samples were purified with a Waters mass-directed purification system utilising a Waters 600 LC pump, Waters Xterra C18 column (5 μm 19 mm×50 mm) and Micromass ZQ mass spectrometer, operating in positive ion electrospray ionisation mode. Mobile phases A (0.1% formic acid in water) and B (0.1% formic acid in acetonitrile) were used in a gradient; 5% B to 100% over 7 min, held for 3 min, at a flow rate of 20 ml/min.

Analytical HPLC

Analytical HPLC was carried out with a Spectra System P4000 pump and Jones Genesis C18 column (4 μm, 50 mm×4.6 mm). Mobile phases A (0.1% formic acid in water) and B (acetonitrile) were used in a gradient of 5% B for 1 min rising to 98% B after 5 min, held for 3 min at a flow rate of 2 ml/min. Detection was by a TSP UV 600OLP detector at 254 nm UV and range 210-600 nm PDA. The Mass spectrometer was a Finnigan LCQ operating in positive ion electrospray mode.

NMR

¹H NMR and ¹³C NMR were recorded using Bruker DPX 300 spectrometer at 300 MHz and 75 MHz respectively. Chemical shifts were reported in parts per million (ppm) on the δ scale relative to tetramethylsilane internal standard. Unless stated otherwise all samples were dissolved in DMSO-d₆.

### Example 1

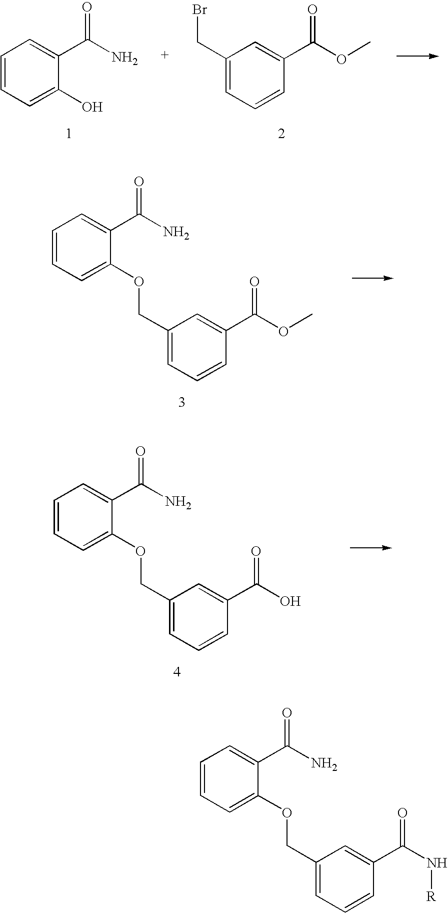

(a) To a suspension of methyl 3-(bromomethyl) benzoate (2) (1.0 g, 7.2 mmol) and potassium carbonate (2.0 g, 14.5 mmol) in acetone (1OmI) was added salicylamide (1) (1.0 g, 7.2 mmol) and the reaction was stirred at 25° C. for 14 hours. The solution was concentrated in vacuo and the resulting residue was treated with water (50 ml) and extracted into dichloromethane (2×50 ml). The combined organic layers was dried with MgSO₄, filtered and concentrated in vacuo to yield a white solid which was purified by column chromatography (50 g silica, hexane: ethyl acetate) to yield 3-(2-carbamoyl-phenoxymethyl)-benzoic acid methyl ester (3) as white solid (2.0 g, 97.60%); m/z [M+1]⁺ 285

(b) To a mixture of 3-(2-carbamoyl-phenoxymethyl)-benzoic acid methyl ester (3) (2.0 g) and 2 M NaOH (4 ml) in methanol (8 ml) was stirred at 25° C. for 14 hours. The solution was concentrated in vacuo. The reaction residue was treated with water (20 ml) and washed with dichloromethane (2×20 ml). The aqueous layer was acidified with 2M HCl, filtered, washed with water and hexane and dried to yield 3-(2-carbamoyl-phenoxymethyl)-benzoic acid (4) as a white solid (1.87 g, 97%); m/z [M+1]⁺ 271, 95% purity

(c) The appropriate amine (0.23 mmol) was added to a solution of 3-(2-carbamoyl-phenoxymethyl)-benzoic acid (4) (0.20 mmol) in dimethylacetamide (1 ml). Hunigs base (0.31 mmol) and 2-(1H-benzotriazole-1-yl)-1,1,3,3-tetramethyluronium hexafluorophosphate (0.25 mmol) were then added and the reaction was stirred at room temperature for 16 hours. The reaction mixtures were then purified by preparative HPLC, to yield the compounds below:

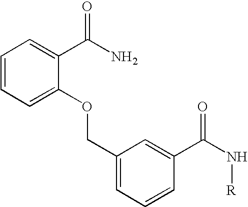

| Compound | R | Purity (%) | RT (min) | [M + H]+ |
|---|---|---|---|---|
| 5 | 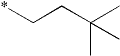 | 90 | 4.28 | 355 |
| 6 | 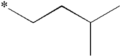 | 90 | 4.13 | 341 |
| 7 | 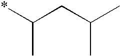 | 90 | 4.29 | 355 |
| 8 | 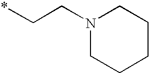 | 90 | 2.98 | 382 |
| 9 | 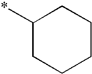 | 90 | 4.11 | 353 |
| 10 | 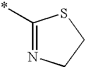 | 90 | 3.34 | 356 |
| 11 | 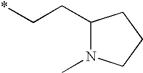 | 90 | 2.98 | 382 |
| 12 | 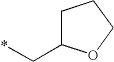 | 90 | 3.5 | 355 |
| 13 | 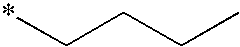 | 90 | 3.9 | 327 |
| 14 | 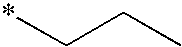 | 90 | 3.66 | 313 |
| 15 | 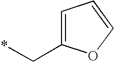 | 90 | 3.8 | 351 |
| 16 | 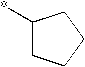 | 90 | 3.91 | 339 |
| 17 | 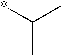 | 90 | 3.65 | 313 |
| 18 | 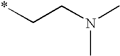 | 90 | | 342 |
| 19 | 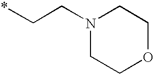 | 90 | 2.9 | 384 |
| 20 | 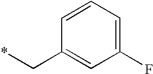 | 90 | 4.08 | 379 |
| 21 | 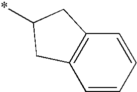 | 90 | | 387 |
| 22 | 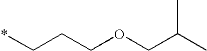 | 90 | 3.92 | 371 |

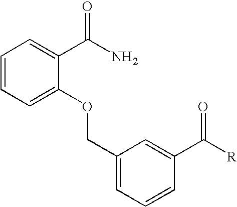

| Compound | R | Purity (%) | RT (min) | [M + H]+ |
|---|---|---|---|---|
| 23 | 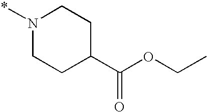 | 90 | 3.88 | 411 |

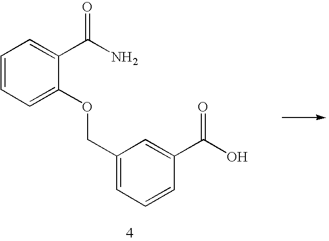

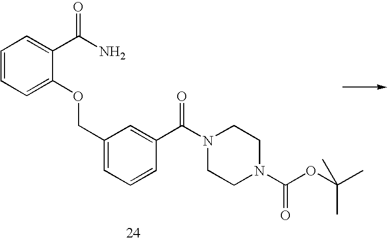

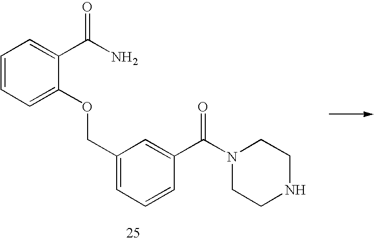

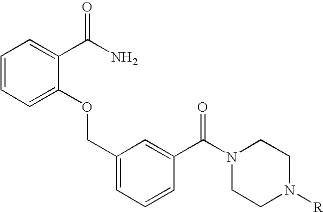

(d) A mixture of 3-(2-carbamoyl-phenoxymethyl)-benzoic acid (4) (0.50 g, 1.8 mmol), Hunigs base (0.48 ml, 2.7 mmol), tert-butyl-1-piperazine carboxylate (0.30 g, 2.0 mmol) and 2-(1H-benzotriazole-1-yl)-1,1,3,3-tetramethyluronium hexafluorophosphate (0.83 g, 2.2 mmol) in dimethylformamide (5 ml) was stirred at 25° C. for 14 hours. The reaction mixture was treated with water (20 ml) and extracted into dichloromethane (2×50 ml). The combined organic layers was washed with brine (50 ml), dried with MgSO₄, filtered and concentrated in vacuo to yield (24) as a yellow oil (1.6 g) which was taken to the next stage without further purification.

(e) A solution of 12 M HCl:ethanol (2:1) was added to 4-[3-(2-carbamoyl-phenoxymethyl)-benzoyl]-piperazine-1-carboxylic acid tert-butyl ester (24) and the reaction was stirred at 25° C. for 14 hours. The reaction was partially concentrated in vacuo and the mixture was diluted with water (50 ml) and basified with ammonia. The reaction mixture was extracted into dichloromethane (2×50 ml). The combined organic layers was washed with brine (50 ml), dried with MgSO₄, filtered and concentrated in vacuo to yield 25 as a white solid (0.6 g); m/z [M+1]⁺ 339, 95% purity

(f) (i) The appropriate isocyanate (0.16 mmol) was added to a solution of 25 (0.15 mmol) in dichloromethane (1 ml). For the sulphonyl chloride reactions, Hunigs base (39 μl, 0.22 mmol) was also added to the reaction mixture. The reactions were stirred at room temperature for 16 hours. The reaction mixtures were then purified by preparative HPLC, to yield the compounds below:

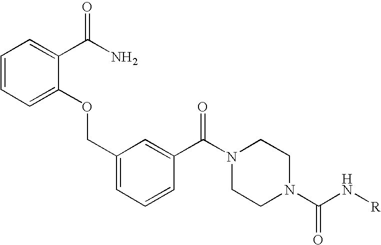

| Compound | R | Purity (%) | RT (min) | [M +H]+ |
|---|---|---|---|---|
| 26 | 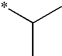 | 95 | 3.5 | 425 |
| 27 | 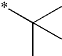 | 95 | 3.75 | 439 |
| 28 | 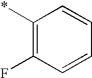 | 90 | 3.83 | 477 |
| 29 | 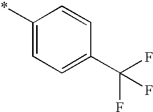 | 85 | 4.31 | 527 |
| 30 | 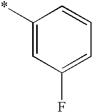 | 95 | 4 | 477 |
| 31 | 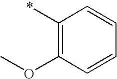 | 95 | 3.94 | 489 |
| 32 | 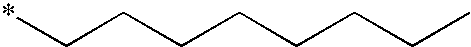 | 95 | 4.59 | 495 |
| 33 | 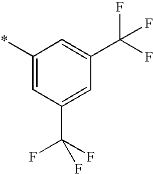 | 95 | 4.82 | 595 |
| 35 | 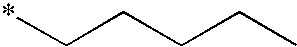 | 95 | 4.19 | 487 |

(ii) The appropriate sulphonyl chloride (0.16 mmol) was added to a solution of 25 (0.15 mmol) in dichloromethane (1 ml). For the sulphonyl chloride reactions, Hunigs base (39 μl, 0.22 mmol) was also added to the reaction mixture. The reactions were stirred at room temperature for 16 hours. The reaction mixtures were then purified by preparative HPLC, to yield the compounds below:

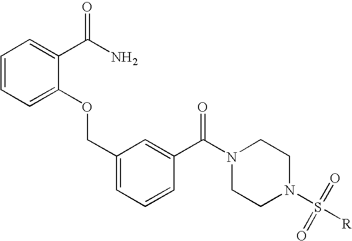

| | | | | [M + |
|---|---|---|---|---|
| Compound | R | Purity (%) | RT (min) | H]+ |
| 36 | 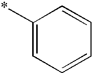 | 95 | 4.08 | 480 |
| 37 | 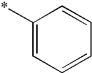 | 95 | 4.66 | 522 |
| 38 | 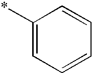 | 90 | 3.78 | 537 |
| 39 | 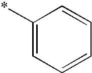 | 95 | 4.08 | 499 |
| 40 | 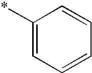 | 95 | 4317 | 498 |
| 41 | 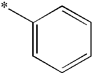 | 80 | 4.14 | 522 |
| 42 | 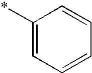 | 95 | 4.09 | 460 |
| 43 | 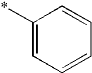 | 95 | 4.08 | 494 |
| 44 | 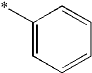 | 95 | 3.65 | 432 |

(iii) The appropriate acid chloride or acid (0.16 mmol) was added to a solution of 25 (0.15 mmol) in dichloromethane (1 ml), followed by Hunigs base (39 μl, 0.22 mmol). 2-(1H-benzotriazole-1-yl)-1,1,3,3-tetramethyluronium hexafluorophosphate (66.8 mg, 0.18 mmol) was then added to all the acid reactions and the reactions were stirred at room temperature for 16 hours. The reaction mixtures were then purified by preparative HPLC, to yield the compounds below:

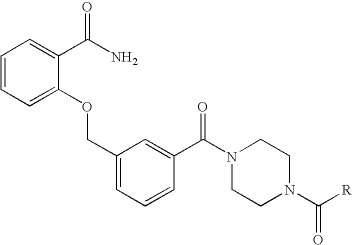

| | | | RT | [M + |
|---|---|---|---|---|
| Compound | R | Purity (%) | (min) | H]+ |
| 45 | 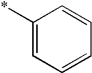 | 95 | 3.73 | 444 |
| 46 | 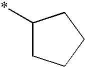 | 95 | 3.83 | 436 |
| 47 | 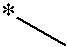 | 95 | 3.26 | 382 |
| 48 | 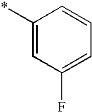 | 95 | 3.8 | 462 |
| 49 | 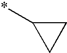 | 95 | 3.47 | 408 |
| 50 | 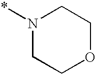 | 95 | 3.4 | 453 |
| 51 | 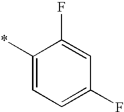 | 95 | 3.86 | 480 |
| 52 | 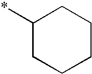 | 95 | 3.95 | 450 |
| 53 | 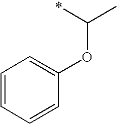 | 95 | 3.98 | 488 |
| 54 | 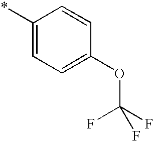 | 95 | 4.23 | 528 |
| 55 | 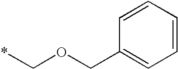 | 95 | 3.87 | 488 |
| 56 | 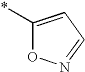 | 95 | 3.53 | 435 |
| 57 | 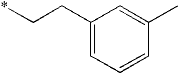 | 90 | 4.09 | 486 |
| 58 | 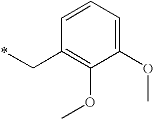 | 95 | 3.86 | 518 |
| 59 | 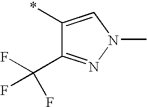 | 95 | 3.66 | 516 |
| 60 | 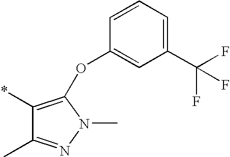 | 90 | 4.08 | 622 |
| 61 | 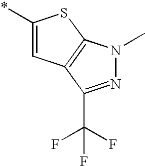 | 95 | 4.17 | 572 |
| 62 | 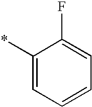 | 95 | 3.73 | 462 |

### Example 2

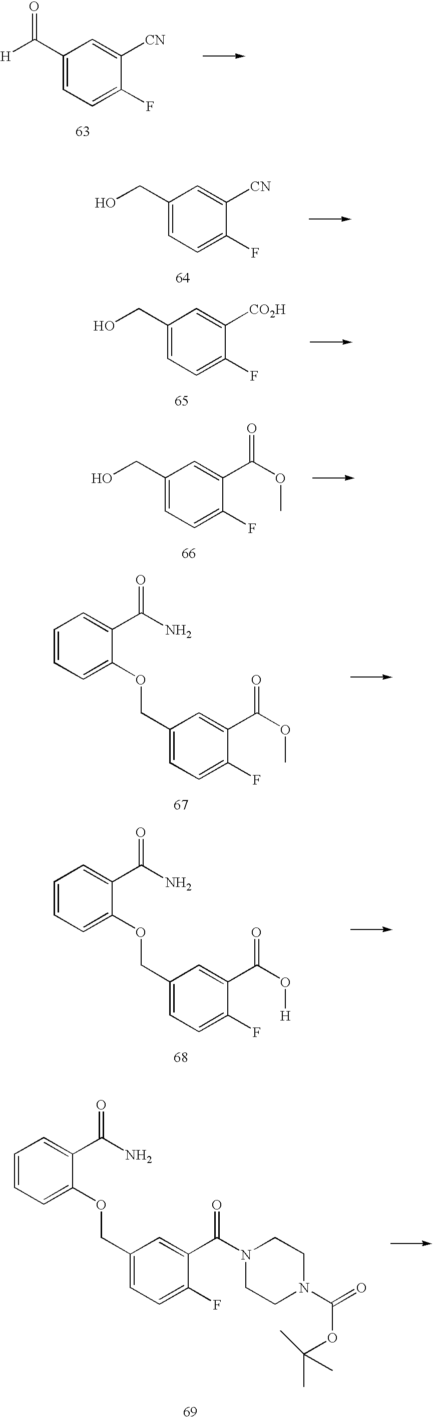

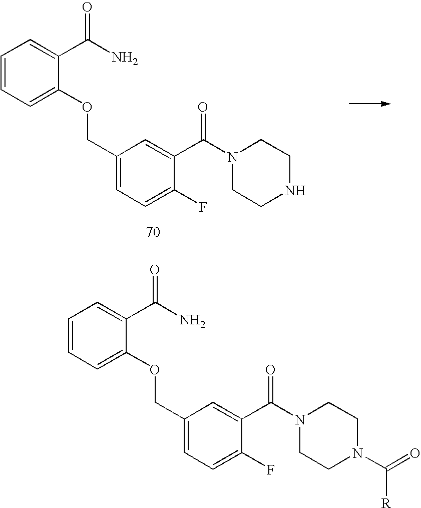

(a) 2-fluoro-5-formylbenzonitrile (63)(10 g, 67.056 mmol) were suspended in 40 ml methanol until complete dissolution. NaBH₄. (2.89 g; 73.76 mmol) was added portionwise over 3 and a half hours. The reaction was stirred at room temperature for 76 hours. Methanol was removed under reduce pressure and the residue was dissolved in DCM (20 ml) to which water (20 ml) was added. The aqueous phase was extracted again with DCM. The organic layers were combined and washed with water, then dried over MgSO₄. DCM was removed under reduce pressure to give compound 64 as a white solid (9.174 g, 95% yield, [M+H]⁺: 152 (weak ionization)).

(b) Compound 64 (7 g, 47 mmol) was dissolved in methanol and sodium hydroxide 5M (20 ml) was added and this was left to stir at 60° C. for 9 hours. The reaction mixture was concentrated in vacuo, the residue taken up in water, and acidified to pH 3 with 6M HCl and a white solid formed. The solid was filtered and the filtrate was concentrated under vacuum. The solid obtained was triturated with toluene, then concentrated under vacuum to azeotrope residual water, and then dried in an oven. The solid obtained, compound 65 (10.77 g, [M−H]⁻: 169) also contained sodium chloride but was used as such in the next step.

(c) Compound 65 (199 g, 113 mmol) was dissolved in methanol before adding concentrated H₂SO₄ (12 ml) slowly, then left to reflux for 18 hours. The reaction mixture was evaporated and sodium bicarbonate (250 ml) was added slowly and extracted with EtOAc (3×150 ml), dried over MgSO₄, concentrated in vacuo. The residue was purified by flash chromatography (eluant: hexane/ethyl acetate 9/1) to give pure compound 66 (9.325 g, [M+H]⁺: 185 (weak ionization)).

(d) Compound 66 (3 g 16.2 mmol) was dissolved in 25 ml acetone, then salicylamide (2.4 g 17.9 mmol) and triphenylphosphine (5.1 g 19.5 mmol) were added. The suspension was stirred at room temperature until all reactants were in solution, then DIAD (3.8 g 19.5 mmol) was added dropwise over 20 mins and the solution left to stir at room temperature for 18 hours. The white precipitate was filtered and recrystallised from hot ethyl acetate to give compound 67 (2.77 g, [M+H]⁺: 304) as a white solid.

(e) Compound 67 (2.7 g, 9 mmol) was suspended in 2M NaOH (10 ml) and 30 ml of methanol. The solution was left to stir at room temperature for 2 hours. The methanol was evaporated and 1 N HCl added until a white solid formed. The solid was filtered and washed with water, then dried to give pure compound 68 (2.5 g, [M+H]⁺: 290).

(f) Compound 69 was synthesised from compound 68 according to the method of Example 1(d) (yield: 72%, [M+H]⁺: 458)

(g) Compound 70 was synthesised from compound 69 according to the method of Example 1(e) ([M+H]⁺: 358)

(i) Using the methods of Example 1 (f)(i), (ii) and (iii) respectively, the following compounds were prepared from 70:

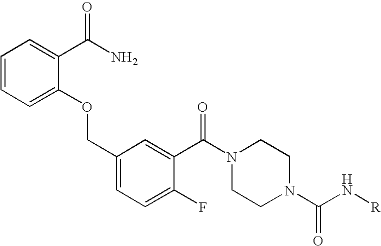

| | | | RT | [M + |
|---|---|---|---|---|
| Compound | R | Purity (%) | (min) | H]+ |
| 72 | 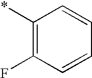 | 85 | 3.76 | 495 |
| 73 | 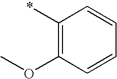 | 95 | 3.83 | 507 |
| 74 | 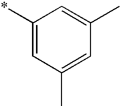 | 95 | 4.07 | 505 |

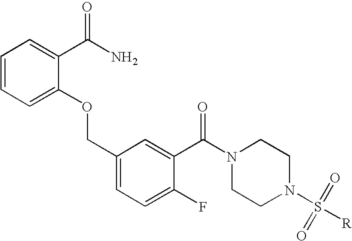

| | | | RT | [M + |
|---|---|---|---|---|
| Compound | R | Purity (%) | (min) | H]+ |
| 75 | 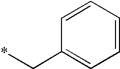 | 80 | 4.01 | 512 |

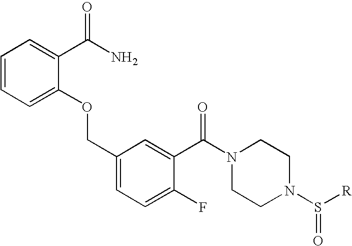

| | | | RT | [M + |
|---|---|---|---|---|
| Compound | R | Purity (%) | (min) | H]+ |
| 76 | 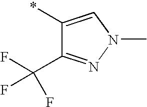 | 85 | 3.65 | 534 |
| 77 | 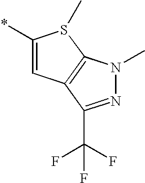 | 90 | 4.11 | 590 |
| 78 | 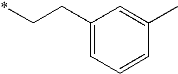 | 95 | 4.07 | 504 |
| 79 | 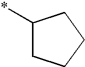 | 95 | 3.78 | 454 |
| 80 | 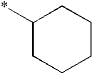 | 90 | 3.93 | 468 |
| 81 | 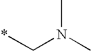 | 87 | 3.39 | 443 |
| 82 | 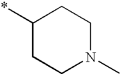 | 99 | 3.38 | 483 |
| 84 | 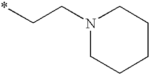 | 61 | 3.43 | 443 |
| 85 | 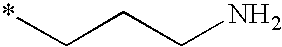 | 81 | 3.46 | 469 |
| 86 | 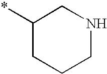 | 75 | 3.42 | 455 |
| 71 | 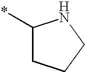 | 90 | 4.42 | 506 |

### Example 3

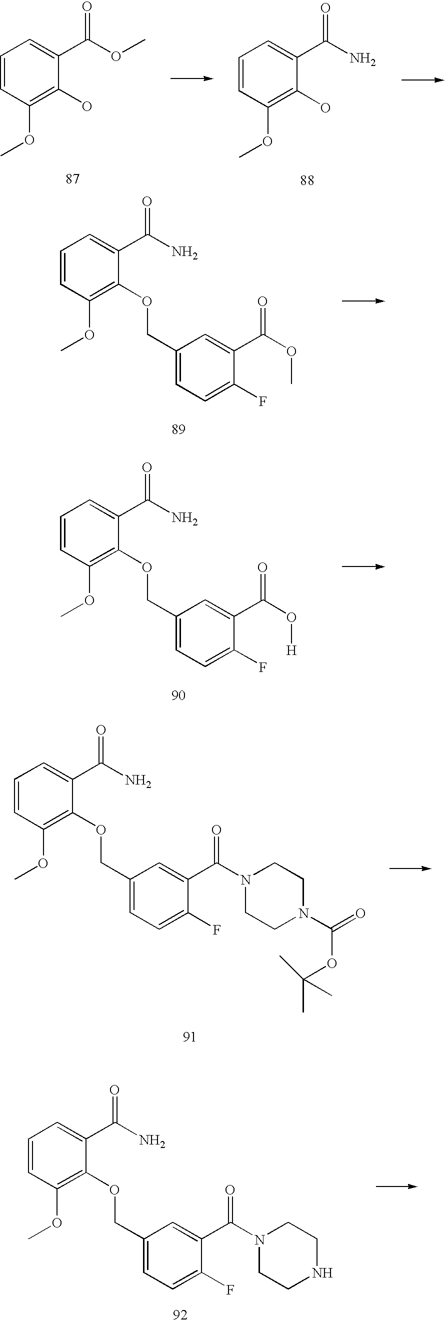

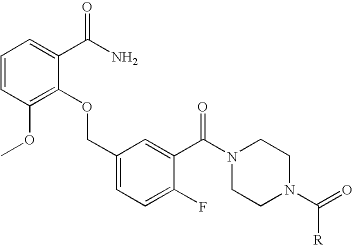

(a) Methyl-3-methoxysalicylate (87)(1.4 g, 7.6 mmol) was suspended in 15 ml of a 7N solution of ammonia in methanol and stirred at 60° C. in a sealed tube for 24 hours. The solution was concentrated under vacuum to yield compound 88 as a brown solid (1.4 g, yield: 93%, [M+H]⁺: 168).

(b) Compound 89 was synthesized from compound 88 according to the method of Example 2(d) (yield: 54%, [M+H]⁺: 334).

(c) Compound 90 was synthesized from compound 89 according to the method of Example 2(e) (yield: 93%, [M+H]⁺: 320).

(d) Compound 91 was synthesized from compound 90 according to the method of Example 1(d) ([M+H]⁺: 488).

(e) Compound 92 was synthesized from compound 91 according to the method of Example 1 (e) ([M+H]⁺: 388).

(f) Using the methods of Example 1 (f)(i), (ii) and (iii) respectively, the following compounds were prepared from 92:

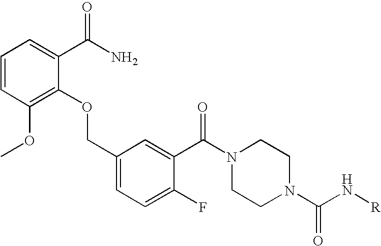

| | | | RT | |
|---|---|---|---|---|
| Compound | R | Purity (%) | (min) | [M + H]+ |
| 93 | 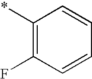 | 94 | 4.52 | 525 |
| 94 | 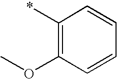 | 77 | 4.66 | 537 |
| 95 | 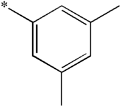 | 92 | 4.97 | 535 |

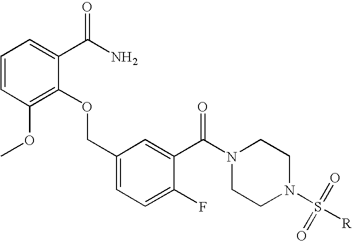

| | | | RT | |
|---|---|---|---|---|
| Compound | R | Purity (%) | (min) | [M + H]+ |
| 96 | 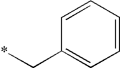 | 92 | 4.91 | 542 |

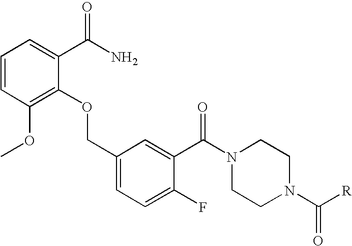

| | | | RT | |
|---|---|---|---|---|
| Compound | R | Purity (%) | (min) | [M + H]+ |
| 97 | 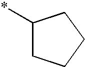 | 80 | 4.59 | 484 |
| 98 | 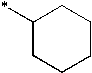 | 88 | 4.77 | 498 |
| 99 | 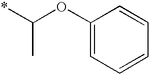 | 88 | 4.77 | 536 |
| 100 | 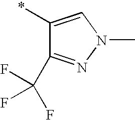 | 81 | 4.45 | 564 |
| 101 | 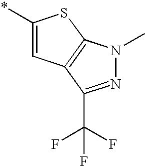 | 93 | 5.03 | 620 |
| 102 | 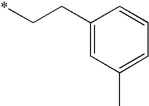 | 97 | 4.99 | 534 |

### Example 4

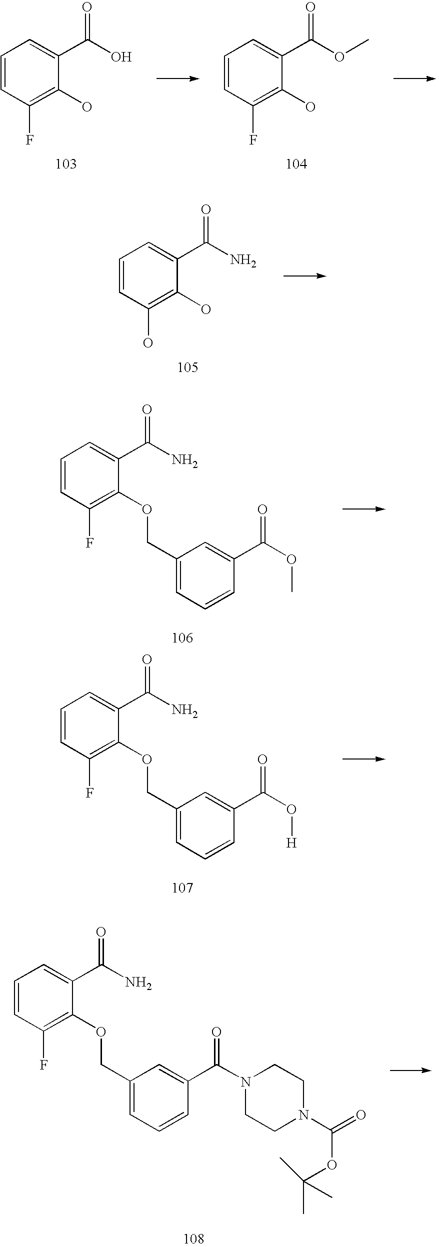

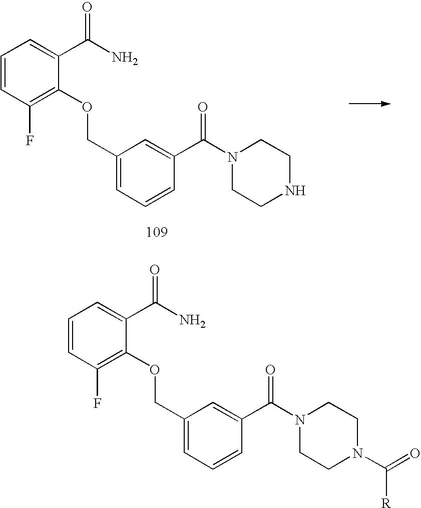

(a) Concentrated sulphuric acid (6 ml) was added to 3-fluoro-2-hydroxybenzoic acid (103)(5 g, 32 mmol) in methanol (20 ml). This was left to stir at reflux 18 hours. The reaction mixture was concentrated under vacuum, then saturated sodium bicarbonate (500 ml) was added and product extracted with EtOAc (3×150 ml). The organic extracts were collated, dried over MgSO₄, and evaporated to give compound 104 (3.9 g, yield: 72%, [M−H]⁻: 169) as liquid which solidified into pale yellow crystals.

(b) Compound 105 was synthesized from compound 104 according to the method of Example 3(a) (yield: 97%, [M+H]⁺: 156).

(c) Compound 106 was synthesized from compound 105 according to the method of Example 2(d) (yield: 80%, [M+H]⁺: 304).

(d) Compound 107 was synthesized from compound 106 according to the method of Example 2(e) ([M+H]⁺: 290).

(e) Compound 108 was synthesized from compound 107 according to the method of Example 1(d)([M+H]⁺: 458).

(f) Compound 109 was synthesized from compound 108 according to the method of Example 1(e) ([M+H]⁺: 358).

(g) Using the methods of Example 1(f)(i), (ii) and (iii) respectively, the following compounds were prepared from 109:

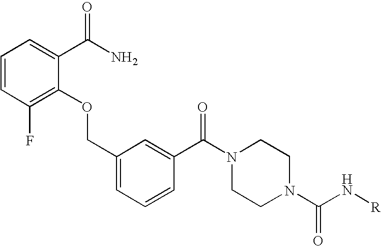

| | | | RT | |
|---|---|---|---|---|
| Compound | R | Purity (%) | (min) | [M + H]+ |
| 110 | 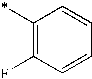 | 88 | 4.54 | 495 |
| 111 | 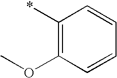 | 88 | 4.67 | 507 |
| 112 | 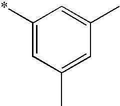 | 94 | 4.98 | 505 |

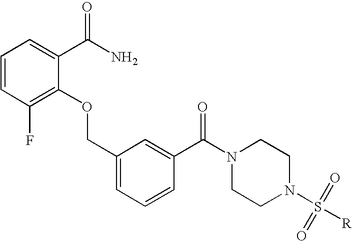

| | | | | |
|---|---|---|---|---|
| | | | RT | |
| Compound | R | Purity (%) | (min) | [M + H]+ |
| 113 | 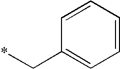 | 98 | 4.9 | 512 |

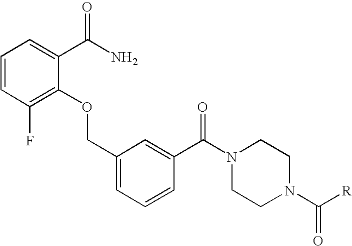

| | | | RT | |
|---|---|---|---|---|
| Compound | R | Purity (%) | (min) | [M + H]+ |
| 114 | 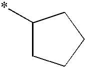 | 92 | 4.57 | 454 |
| 115 | 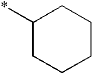 | 92 | 4.74 | 467 |
| 116 | 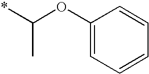 | 95 | 4.76 | 506 |
| 117 | 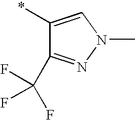 | 94 | 4.46 | 534 |
| 118 | 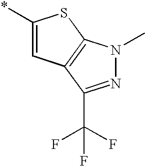 | 95 | 5.02 | 590 |
| 119 | 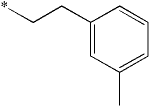 | 99 | 4.97 | 504 |

### Example 5

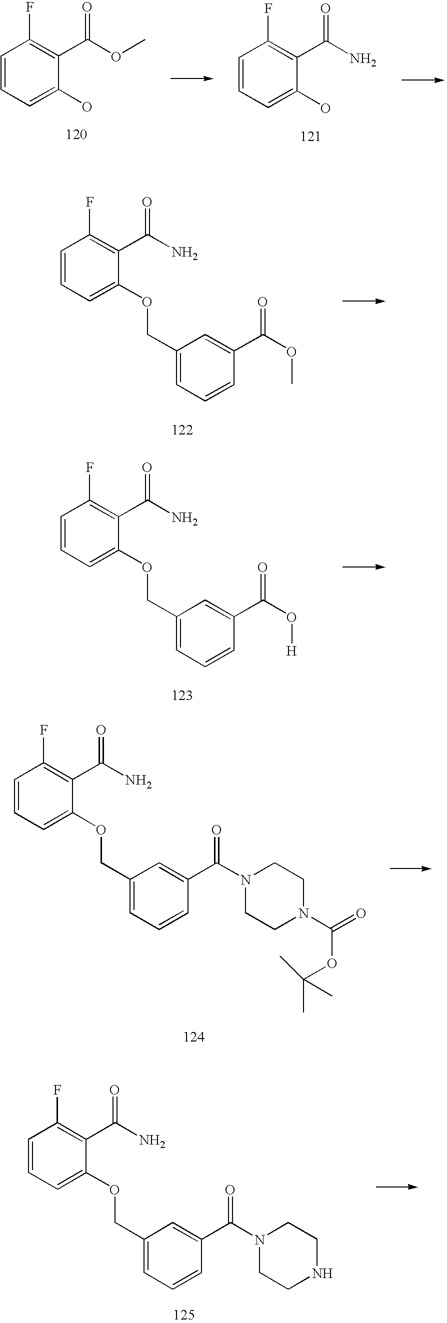

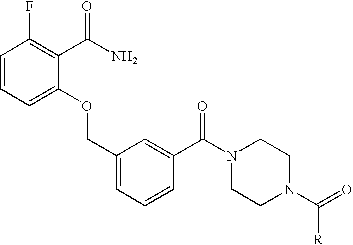

(a) Compound 121 was synthesized from compound 120 according to the method of Example 3(a) (yield: 93%, [M+H]⁺: 156).

(b) Compound 122 was synthesized from compound 121 according to the method of Example 2(d)([M+H]⁺: 304).

(c) Compound 123 was synthesized from compound 122 according to the method of Example 2(e)([M−H]⁻: 288).

(d) Compound 124 was synthesized from compound 123 according to the method of Example 1 (d)([M+H]⁺: 458).

(e) Compound 125 was synthesized from compound 124 according to the method of Example 1(e)([M+H]⁺: 358).

(f) Using the methods of Example 1(f)(i), (ii) and (iii) respectively, the following compounds were prepared from 125:

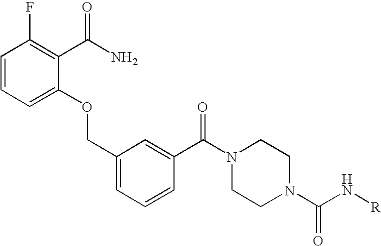

| | R | Purity (%) | RT (min) | [M +H]+ |
|---|---|---|---|---|
| 126 | 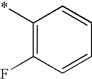 | 95 | 4.43 | 494 |
| 127 | 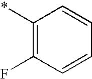 | 100 | 4.56 | 507 |
| 128 | 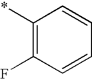 | 99 | 4.88 | 505 |

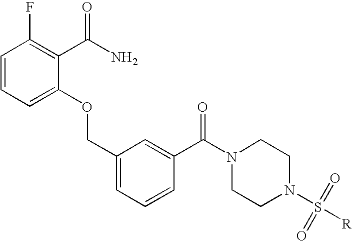

| | R | Purity (%) | RT (min) | [M +H]+ |
|---|---|---|---|---|
| 129 | 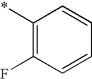 | 99 | 4.82 | 512 |

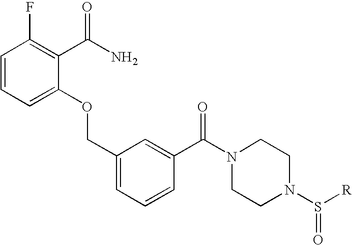

| | R | Purity (%) | RT (min) | [M +H]+ |
|---|---|---|---|---|
| 130 | 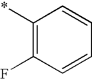 | 100 | 4.44 | 454 |
| 131 | 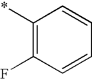 | 100 | 4.62 | 468 |
| 132 | 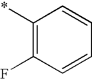 | 100 | 4.86 | 504 |
| 133 | 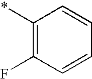 | 83 | 4.34 | 534 |
| 134 | 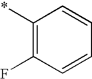 | 100 | 4.91 | 590 |
| 135 | 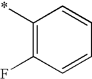 | 100 | 4.66 | 506 |

### Example 6

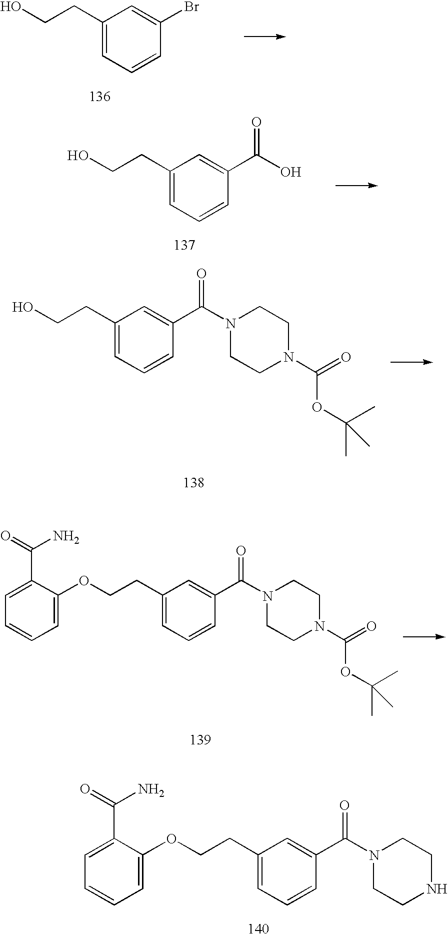

(a) To a cooled (−78° C.) solution of 2-(3-bromo-phenyl)-ethanol (136) (15.0 g, 74.6 mmol) in anhydrous diethyl ether (200 ml) was added N,N,N′,N′, tetremethylethylenediamine, (TMEDA) (22.2 ml, 149.2 mmol). After 5 minutes of stirring at -78° C., n-butyl lithium (2.5 M in hexanes; 59.7 ml, 149.2 mmol) was added dropwise over a period of 10 minutes, (a white precipitate formed approximately half way through the addition). The temperature was allowed to rise up to −20° C. for over 1 hour, and then taken to −78° C. again. Dry carbon dioxide gas was then bubbled through the reaction mixture for 10 minutes until the exotherm had ceased and then allowed to warm to room temperature over 1 hour. The ether was extracted with water (115 ml). The aqueous layer was then acidified with HCl (6N) to pH 0.5. The resulting white precipitate was then extracted with ethyl acetate (2×170 ml). The combined organics were dried over magnesium sulfate, filtered and concentrate in vacuo to afford 137 as a buff white powder. Single peak in LC-MS analysis. (11.40 g, 92%) and required no further purification. m/z (LC-MS, ESN), RT=3.21 min, (M−H)=165.0.

¹H NMR (300 MHz, D⁶-DMSO): 12.88(1H, —COOH), 7.86 (1H, S) 7.83 (1H, dt J 2.1 Hz, J 7.5 Hz), 7.53 (1H, d, J 2.1 Hz), 7.46 (1H, t, J 7.5 Hz), 4.68(1H, —OH), 3.68 (2H, t, J 6.7 Hz), 2.84 (2H, t, J 6.7 Hz).

(b) To a solution of 3-(2-hydroxy-ethyl)-benzoic acid (137)(12.0 g , 72 mmol) in DCM (150 mL) was added tert-butyl 1-piperazinecarboxylate (14.9 g, 80.0 mmol) & O-benzotriazole-N,N,N′,N′-tetramethyl-uronium-hexafluoro-phosphate (30.2 g, 80.0 mmol). The mixture was stirred for 5 minutes before triethylamine (20.6 ml, 150.0 mmol) was added. After 30 minutes of stirring at room temperature the reaction mixture was filtered, and the concentrated in vacuo. The resultant oil was subjected to chromatography using ethyl acetate: hexane 1:1 (rf 0.13), a white solid 138 was isolated. Single peak in LC-MS analysis. (18.0 g, 75%) and required no further purification. m/z (LC-MS, ESP), RT=3.79 min. (M+H) 334.

(c) (i) To a cooled solution of 4-[3-(2-Hydroxy-ethyl)-benzoyl]-piperazine-1-carboxylic acid tert-butyl ester (138)(10.0 g, 29.9 mmol) in DCM (100 ml) at −5° C., was added dropwise triethylamine (5 mL, 35.9 mmol) followed by methane sulphonyl chloride (2.8 mL, 35.8 mmol), allowing the reaction to warm to room temperature over 30 minutes. The mixture was then washed with water (2×25 ml). The organic layer was washed was dried over MgSO₄, filtered and concentrated to afford an oil. LC-MS analysis. (9.75 g 79% yield) and required no further purification. m/z (LC-MS, ESP), RT=4.11 mins. (M+H)=413.

(ii) To the crude oil isolated (5.8 g, 22.5 mmol) in (i) was dissolved in dimethyl formamide (50 mL) followed by cesium carbonate (7.3 g,22.4 mmol)and salicylamide (1)(3.08 g, 22.4 mmol). The reaction was the cooled in a fridge overnight and washed with (2×50 mL) of water, followed by hexane (2×50 mL) of finally TBME (2×50 ml). The resulting white solid 139 was dried at room temperature under vacuum overnight. Single peak in LC-MS analysis. (6.3 g, 95% purity) and required no further purification. m/z (LC-MS, ESP), RT=4.13 mins. (M+H) 413.

(d) To 4-{3-[2-(2-carbamoyl-phenoxy)-ethyl]-benzoyl}-piperazine-1-carboxylic acid tert-butyl ester (139)(4.2 g, 9.2 mmol) was added 4M hydrogen chloride in dioxane (14.4 mL, 57.0 mmol). After 15 minutes the solvent was removed in vacuo and 7M ammonia in methanol (15 mL, 75 mmol) added. The resultant cream precipate was filtered and filtrate concentrated in vacuo to afford a sticky gum 140(2.9 g, 90% yield). LC-MS analysis. >95% purity) no further purification attempted. m/z (LC-MS, ESP), RT=3.10 mins. (M+H)=354

(e) Using the methods of Example 1(f)(i), (ii) and (iii) respectively, the following compounds were prepared from 140:

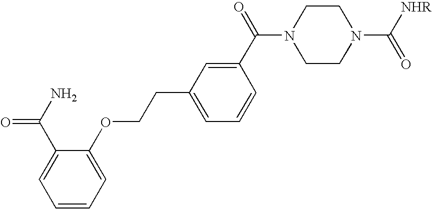

| | | | RT | |
|---|---|---|---|---|
| Compound | R | Purity (%) | (min) | [M + H]+ |
| 141 | 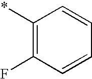 | 99 | 4.79 | 491 |
| 142 | 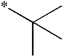 | 100 | 4.55 | 452.6 |
| 143 | 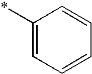 | 100 | 4.65 | 472.5 |
| 144 | 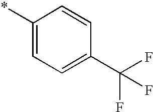 | 100 | 5.23 | 540.5 |
| 145 | 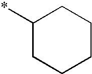 | 99 | 9.83 | 478.6 |
| 146 | 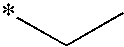 | 98 | 4.11 | 424.5 |
| 147 | 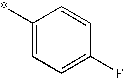 | 96 | 9.83 | 490.5 |
| 148 | 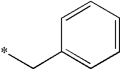 | 99 | 9.49 | 486.6 |

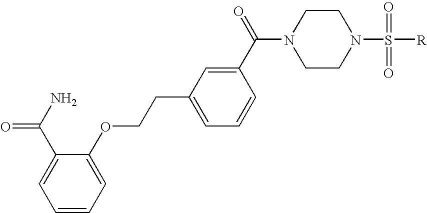

| | | | RT | |
|---|---|---|---|---|
| Compound | R | Purity (%) | (min) | [M + H]+ |
| 149 | 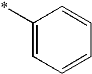 | 98 | 10.43 | 493.6 |
| 150 | 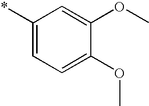 | 99 | 10.17 | 553.6 |
| 151 | 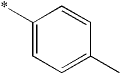 | 100 | 11.10 | 507.6 |
| 152 | 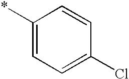 | 100 | 11.59 | 528.0 |
| 153 | 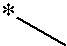 | 99 | 8.21 | 431.5 |
| 154 | 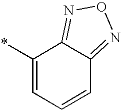 | 100 | 10.71 | 535.6 |
| 155 | 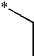 | 98 | 8.66 | 445.5 |
| 156 | 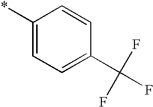 | 100 | 12.21 | 561.6 |
| 157 | 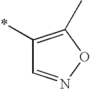 | 84 | 7.60 | 498.6 |
| 158 | 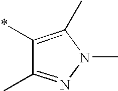 | 99 | 9.26 | 525.6 |
| 159 | 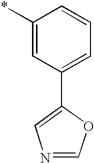 | 98 | 10.25 | 560.6 |
| 160 | 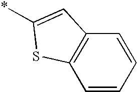 | 99 | 12.20 | 549.7 |
| 161 | 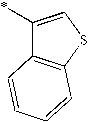 | 85 | 11.80 | 549.7 |
| 162 | 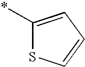 | 99 | 10.36 | 499.6 |
| 163 | 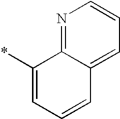 | 99 | 10.12 | 544.6 |

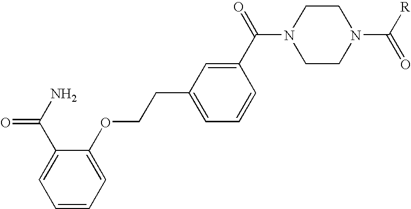

| | | | RT | |
|---|---|---|---|---|
| Compound | R | Purity (%) | (min) | [M + H]+ |
| 164 | 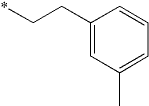 | 99 | 5.21 | 500 |
| 165 | 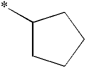 | 97 | 4.82 | 450 |
| 166 | 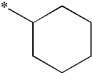 | 98 | 4.99 | 464 |
| 167 | 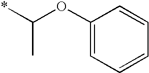 | 98 | 5.00 | 502 |
| 168 | 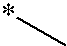 | 94 | 7.32 | 395.5 |
| 169 | 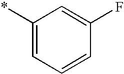 | 100 | 4.64 | 475.5 |
| 170 | 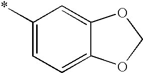 | 100 | 9.22 | 501.5 |
| 171 | 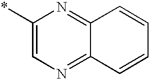 | 97 | 9.30 | 509.6 |
| 172 | 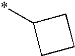 | 100 | 4.46 | 435.5 |
| 173 | 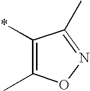 | 100 | 4.30 | 474.6 |
| 174 | 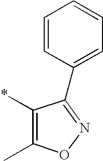 | 99 | 10.14 | 538.6 |
| 175 | 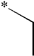 | 99 | 4.16 | 409.5 |
| 176 | 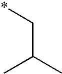 | 100 | 9.14 | 437.5 |
| 177 | 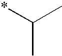 | 91 | 8.42 | 423.5 |
| 178 | 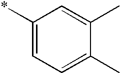 | 97 | 10.46 | 485.6 |

### Example 7

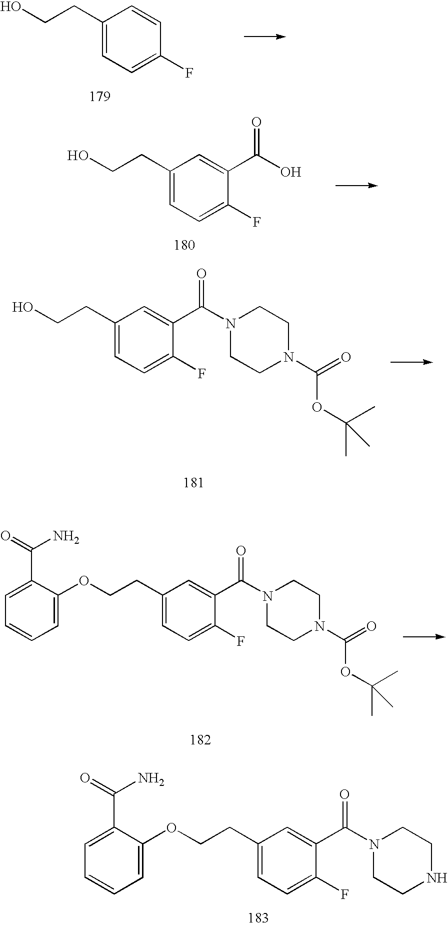

(a) To a solution of 2,2-tetramethtylpiperdine (179)(10.7 mL, 64.0 mmol) in dry tetrahydrofuran (50 mL) was added to a cooled solution of 2.5M n-butyl lithium (26.5 mL, 64.0 mmol) in hexanes/THF at −75° C. 2-(4-fluoro-phenyl)-ethanol (4.5 9, 32 mmol) was then added dropwise to the reaction mixture, maintaining the temperature at −75° C. After 6 hours of stirring under nitrogen a orange red solution resulted. Carbon dioxide gas was bubbled through the reaction mixture over a period of 15 minutes. The reaction was then warmed to room temperature and the organics reduced in vacuo. The residue was taken up into water (40 ml) and washed with DCM (25 ml×2). The pH of the aqueous phase was then adjusted to pH 1 using dilute hydrochloric acid (100 ml, 1 N). The solution was then extracted with ethyl acetate (3×50 mL). The combined organics were dried over magnesium sulfate and concentrated to provide a crude waxy solid. The solid was recrystallized from ethyl acetate to yield 180 as a white crystalline solid. LC-MS analysis. (2.4 g, 95% purity) and required no further purification. m/z (LC-MS, ESN), RT=2.66 mins. (M−H)=183. ¹H NMR (300 MHz, D⁶-DMSO): 14.30 (1H, —COOH), 7.70 (1H, dd, J 2.1, 7.2 Hz), 7.47 (1H, ddd J 2.1, 6.0, 8.4 Hz), 7.20 (1H, dd, J 8.5, 11.1 Hz), 4.59 (1H, —OH), 3.60 (2H t, J 6.9 Hz), 2.74 (2H, t, J 6.9 Hz).

(b) To a solution of 2-fluoro-5-(2-hydroxy-ethyl)-benzoic acid (180)(2.5 g ,15 mmol) in DCM (50 mL) was added tert-butyl 1-piperazinecarboxylate (3.09 g, 16.6 mmol) and O-Benzotriazole-N,N,N′,N′-tetramethyl-uronium-hexafluoro-phosphate (6.27 g, 16.6 mmol). The mixture was stirred for 5 minutes before triethylamine (5.8 mL, 33.1 mmol) was added. After a further 30 minutes of stirring at room temperature the reaction mixture was filtered, and the concentrated in vacuo. The resultant oil was subjected to chromatography using ethyl acetate: hexane 9:1 (rf 0.25). A white solid 181 was isolated. Single peak in LC-MS analysis. (3.97 g, 75%) and required no further purification. m/z (LC-MS, ESP), RT=3.28 min. (M+H)=353.

(c) (i) To a cooled solution of (181) (2.5 g, 13.6 mmol) in DCM (30 ml) at −5° C., was added dropwise triethylamine (2.1 mL, 14 mmol) followed by methane sulphonyl chloride (1.61 g, 14 mmol), allowing the reaction to warm to room temperature over 45 minutes. The mixture was then washed with water (2×15 ml). The organic layer was washed was dried over MgSO₄, filtered and concentrated to afford an oil, (2.6 g 82% yield) and required no further purification. m/z (LC-MS, ESP), RT=4.46 mins. (M+H)=431.

(ii) To the crude oil isolated (2.5 g, 5.8 mmol) in part (i) was dissolved in dimethyl formamide (15mL) followed by cesium carbonate (7.3 g, 5.9 mmol) and salicylamide (802mg, 5.9 mmol). The reaction was the cooled in a fridge overnight and washed with (2×10 mL) of water, followed by hexane (2×10 mL) of finally TBME (10 ml). The resulting white solid (182) was dried RT under vacumn Overnight. Single peak in LC-MS analysis. (1.9 g, 65% purity) and required no further purification. m/z (LC-MS, ESP), RT=3.56 mins, (M+H) 472. ¹H NMR (300 MHz, D₆-DMSO) 7.78 (1H, dd J=1.8, 7.8 Hz), 7.49-7.42 (2H, m), 7.37 (1H, dd, J=2.1, 6.6 Hz), 7.2 (2H, m), 7.01 (1H, m), 4.37 (2H,m), 3.59-3.62 (2H,m), 3.39-3.40 (2H, m), 3.24-3.27 (2H,m), 3.12-2.19 (4H,m), 2.40 (9H,s).

(d) To 4-{5-[2-(2-Carbamoyl-phenoxy)-ethyl]-2-fluoro-benzoyl}-piperazine-1-carboxylic acid tert-butyl ester (182)(0.472 g, 1.0 mmol) was added 4M hydrogen chloride in dioxane (3.0 mL, 10.0 mmol). After 15 minutes the solvent was removed in vacuo and 7M ammonia in methanol (2 mL, 13 mmol) added. The resultant cream precipate was filtered and filtrate concentrated in vacuo to a white foam 183 (0.31 g 84% yield). LC-MS analysis. >90% purity) no further purification attempted. m/z (LC-MS, ESP), RT=2.52 mins. (M+H)=372.

(e) Using the methods of Example 1(f)(i) and (iii) respectively, the following compounds were prepared from 183:

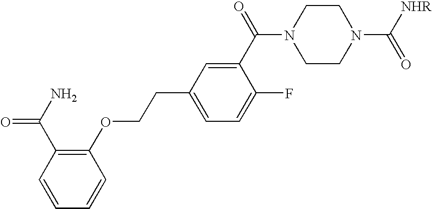

| | | | RT | |
|---|---|---|---|---|
| Compound | R | Purity (%) | (min) | [M + H]+ |
| 184 | 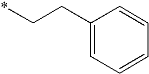 | 98 | 4.83 | 519 |

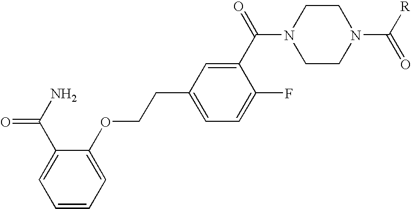

| | | | RT | |
|---|---|---|---|---|
| Compound | R | Purity (%) | (min) | [M + H]+ |
| 185 | 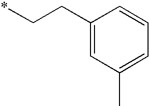 | 99 | 5.15 | 518 |
| 186 | 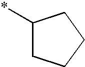 | 99 | 4.76 | 468 |
| 187 | 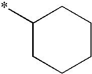 | 99 | 4.93 | 482 |
| 188 | 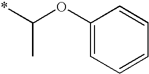 | 90 | 4.94 | 520 |
| 189 | 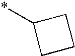 | 92 | 4.57 | 454 |
| 190 | 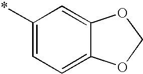 | 99 | 4.64 | 520 |
| 191 | 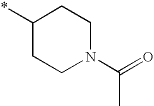 | 96 | 3.36 | 525 |

### Example 8

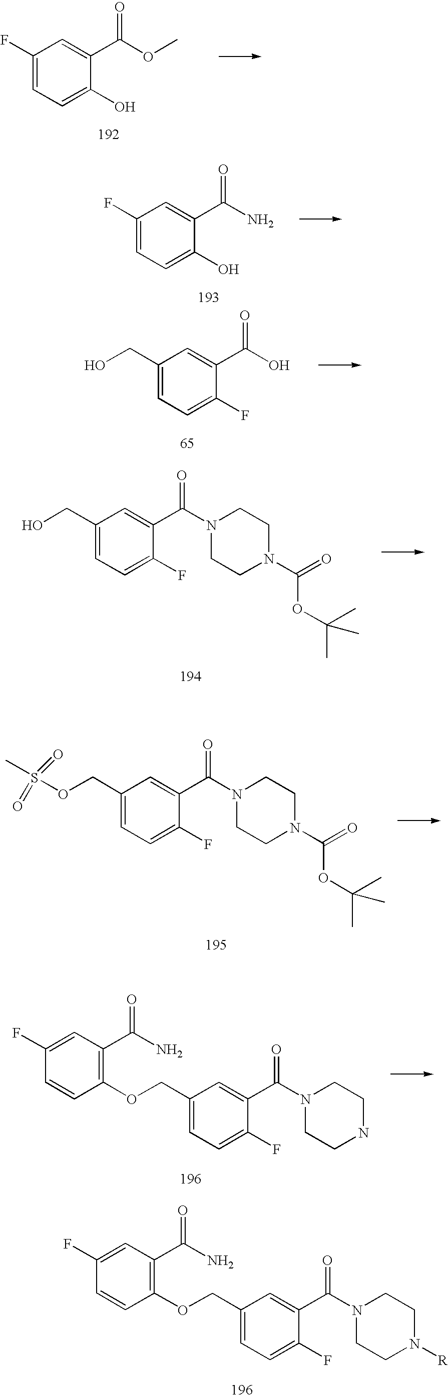

(a) 5-Fluoro-2-hydroxy-benzamide (193)

To a screw tight 50 mL pressure vessel was added methyl 5-fluoro-2-hydroxybenzoate (1.0 g, 5.88 mmol) and (7N) ammonia in methanol (15 ml). The pressure vessel was sealed and contents stirred over night at 60° C. The reaction was cooled to room temperature and the solution evapourated to dryness to afford white crystalline solid. Single peak in LC-MS (0.91 g, 100% purity); m/z (LC-MS, ESP), RT=2.94 mins, (M+H) 156.

(b) 4-(2-Fluoro-5-hydroxymethyl-benzoyl)-piperazine-1-carboxylic acid tert-butyl ester (194)

To 2-fluoro-5-hydroxymethyl-benzoic acid (65)(8.50 g, 50.0 mmol) in DMF (90 mL) at 20° C. was added triethyl amine (13.8 mL, 100 mmol), followed by piperazine-1-carboxylic acid tert-butyl ester (11.16 g, 60.0 mmol), then HBTU (24.6 g, 65.0 mmol) added portionwise over 15 mins, a slight exotherm was noted, the reaction was stirred 30 mins. The reaction mixture was then cooled to 15° C., water (100 mL) added dropwise, forming an sticky yellow suspension. The aqueous liquor was extracted into DCM (3×80 mL), extracts washed with dilute sodium carbonate solution (100 mL), water (100 mL),dried over sodium sulfate, passed through a thin silica pad. Filtrate concentrated under vacuum to afford a colourless oil. Single peak in LC-MS (15.4 g, 91% yield) and taken through to next step without need for any purification; m/z (LC-MS, ESP), RT=3.84 mins, (M+H) 339.

(c) 4-(2-Fluoro-5-methanesulfonyloxymethyl-benzoyl)-piperazine-1-carboxylic acid tert-butyl ester (195)

To a solution of 4-(2-fluoro-5-hydroxymethyl-benzoyl)-piperazine-1 carboxylic acid tert-butyl ester (194)(6.8 g, 20.12 mmol) in dry DCM (60 mL) was added thriethyl amine (2.7 mL, 20.12 mmol). The resulting solution was cooled to 5° C., methane sulfonyl chloride (1.55 mL, 20.12 mmol) was added dropwise over 5 minutes. After 30 minutes the reaction was washed with water (2×50 mL) and dried over sodium sulfate to afford a tacky glass. Single peak in LC-MS (6.37 g, 76% yield) and taken through to next step without need for any purification; m/z (LC-MS, ESP), RT=4.20 mins, (M+H) 417.

(d) 4-[5-(2-Carbamoyl-4-fluoro-phenoxymethyl)-2-fluoro-benzoyl]-piperazine-1-carboxylic acid tert-butyl ester (196)

To a solution of 4-(2-fluoro-5-methanesulfonyloxymethyl-benzoyl)-piperazine-1-carboxylic acid tedt-butyl ester (195)(0.832 g, 2.0 mmol) in DMF (3mL) under a nitrogen blanket was added 5-fluoro-2-hydroxy-benzamide (193)(0.31 g, 2.0 mmol), followed by potassium carbonate (0.552 g, 4.0 mmol). The mixture was then heated to 90° C. After 1 hour of heating the reaction was cooled to 45° C. water (4 mL) was added. The reaction was then cooled to 0° C. with stirring. A fine white suspension resulted. The solid was filtered, washed with cold water (2×10 mL), hexane (2×10 mL) and TBME (2×10 ml). The dried solid provided single peak in LC-MS (0.548 g, 57% yield) and taken through to next step without need for any purification; m/z (LC-MS, ESP), RT=3.18 mins, (M+H) 476.

(e) 5-Fluoro-2-[4-fluoro-3-(piperazine-1-carbonyl)-benzyloxy]-benzamide (197)

To a solution of conc HCl (15 mL) in ethanol (7 mL) was added portionwise 4-[5-(2-carbamoyl-4-fluoro-phenoxymethyl)-2-fluoro-benzoyl]-piperazine-1-carboxylic acid tert-butyl ester (196)(3.49 g, 7.35 mmol). After 1 hour the reaction mixture was concentrated in vacuo and the aqueous residue was diluted with water (50 mL), washed with ether (2×30 mL), and then basified with aqueous ammonia (5 mL) and then extracted with ethyl acetate (3×50 mL). The combined extracts were dried over sodium sulfate and concentrated in vacuo to afford a crystalline solid. Single peak in LC-MS (2.73 g, 98% yield) and taken through to next step without need for any purification; m/z (LC-MS, ESP), RT=3.05 mins, (M+H) 376.

(f) Library Compounds

(i) The appropriate isocyanate (0.15 mmol) was added to a solution of the appropriate 5-fluoro-2-[4-fluoro-3-(piperazine-1-carbonyl)-benzyloxy]-benzamide (197)(0.20 mmol) in dichloromethane (2 mL). The reaction was stirred at room temperature for 16 hours. The reaction mixtures were then purified by preparative HPLC, to yield the compounds below:

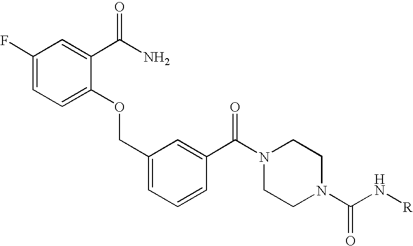

| Patent | R | Purity (%) | RT (min) | [M + H]+ |
|---|---|---|---|---|
| 199 | 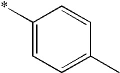 | 99 | 4.03 | 509 |
| 200 | 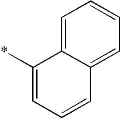 | 93 | 5.06 | 544 |
| 201 | 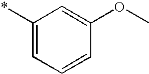 | 99 | 4.94 | 525 |
| 202 | 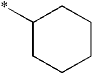 | 98 | 4.77 | 501 |
| 203 | 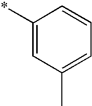 | 99 | 5.03 | 509 |
| 204 | 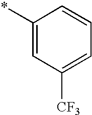 | 99 | 5.39 | 563 |
| 205 | 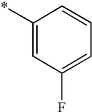 | 99 | 4.82 | 513 |
| 206 | 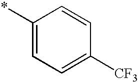 | 99 | 5.4 | 563 |
| 207 | 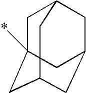 | 100 | 5.57 | 553 |
| 208 | 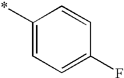 | 100 | 4.96 | 513 |
| 209 | 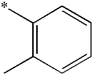 | 100 | 4.87 | 509 |
| 210 | 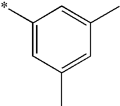 | 100 | 5.18 | 523 |
| 211 | 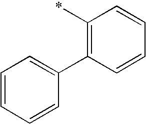 | 99 | 5.23 | 571 |
| 212 | 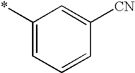 | 100 | 4.91 | 520 |
| 213 | 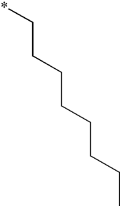 | 100 | 5.75 | 531 |
| 214 | 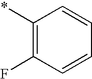 | 99 | 4.82 | 513 |

(ii) The appropriate sulfonyl chloride (0.1 mmol) was added to a solution of 5-fluoro-2-[4-fluoro-3-(piperazine-1carbonyl)-benyloxy]-benzamide (197)(0.1 mmol) in dichloromethane (1.5 mL) together with triethylamine (0.2 mmol). The reactions were stirred overnight and then purified by preparative HPLC, to yield the compounds below:

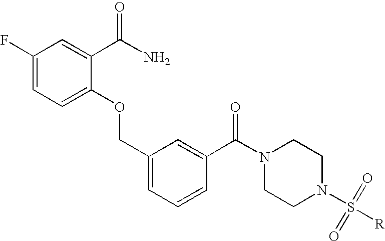

| Patent | R | Purity (%) | RT (min) | [M + H]+ |
|---|---|---|---|---|
| 215 | 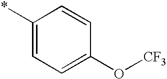 | 100 | 5.52 | 599 |

(iii) The appropriate acid chloride (0.1 mmol) was added to a solution of 5-fluoro-2-[4-fluoro-3-(piperazine-1carbonyl)-benyloxy]-benzamide (197)(0.1 mmol) in dichloromethane (1.5 mL) together with triethylamine (0.2 mmol). The reactions were stirred overnight and were then purified by preparative HPLC, to yield the compounds below:

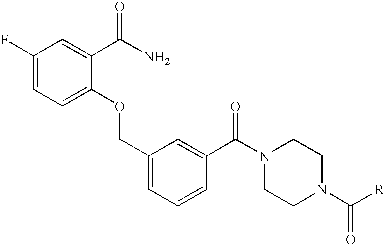

| Patent | R | Purity (%) | RT (min) | [M +H]+ |
|---|---|---|---|---|
| 216 | 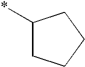 | 100 | 4.71 | 472 |
| 217 | 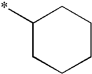 | 100 | 4.88 | 486 |

### Example 9

In order to assess the inhibitory action of the compounds, the following assay was used to determine IC₅₀ values or percentage inhibition at a given concentration.

Mammalian PARP, isolated from Hela cell nuclear extract, was incubated with Z-buffer (25 mM Hepes (Sigma); 12.5 mM MgCl₂ (Sigma); 50 mM KCl (Sigma); 1 mM DTT (Sigma); 10% Glycerol (Sigma) 0.001% NP-40 (Sigma); pH 7.4) in 96 well FlashPlates (TRADE MARK) (NEN, UK) and varying concentrations of said inhibitors added. All compounds were diluted in DMSO and gave final assay concentrations of between 10 and 0.01 μM, with the DMSO being at a final concentration of 1% per well. The total assay volume per well was 40 μl.

After 10 minutes incubation at 30° C. the reactions were initiated by the addition of a 10 μl reaction mixture, containing NAD (5 μM), ³H-NAD and 30mer double stranded DNA-oligos. Designated positive and negative reaction wells were done in combination with compound wells (unknowns) in order to calculate % enzyme activities. The plates were then shaken for 2 minutes and incubated at 30° C. for 45 minutes.

Following the incubation, the reactions were quenched by the addition of 50 μl 30% acetic acid to each well. The plates were then shaken for 1 hour at room temperature.

The plates were transferred to a TopCount NXT (TRADE MARK) (Packard, UK) for scintillation counting. Values recorded are counts per minute (cpm) following a 30 second counting of each well.

The % enzyme activity for each compound is then calculated using the following equation:

$$\% Inhibition = 100 - ( 100 \times \frac{( cpm of unknowns - mean negative cpm )}{( mean positive cpm - mean negative cpm )} )$$

IC₅₀ values (the concentration at which 50% of the enzyme activity is inhibited) were calculated, which are determined over a range of different concentrations, normally from 10 μM down to 0.001 μM. Such IC₅₀ values are used as comparative values to identify increased compound potencies.

The following compounds have a IC₅₀ of less than 0.1 μM: 29, 35, 37, 43, 53, 71, 72, 73, 74, 75, 77, 78, 79, 80, 86, 141, 164, 174, 185, 187, 188, 198, 199, 200, 201, 202, 203, 204, 205, 206, 207, 208, 209, 210, 211, 212, 213, 214, 215, 216, 217.

In addition to the above, the following compounds have an IC₅₀ of less than 0.5 μM: 28, 30, 31, 32, 33, 34, 39, 41, 42, 44, 45, 46, 48, 50, 51, 52, 54, 55, 56, 57, 58, 59, 61, 62, 76, 81, 82, 83, 84, 85, 128, 129, 135, 143, 144, 145, 147, 148, 152, 158, 159, 160, 161, 166, 167, 184, 186, 189, 191.

In addition to those above, the following compounds have an IC₅₀ of less than 1 μM: 15, 26, 27, 36, 38, 40, 47, 49, 60, 116, 190.

In addition to those above, the following compounds have an IC₅₀ of less than 10 μM: 5, 6, 8, 9, 10, 12, 13, 14, 17, 18.

No IC₅₀ value was determined for the following compounds, but they exhibit an inhibition at 1.5 μM of 25% or greater: 97, 99, 110, 111, 112, 113, 126, 127, 130, 131, 132, 133, 134.

The Potentiation Factor (PF₅₀) for compounds is calculated as a ratio of the IC₅₀ of control cell growth divided by the IC₅₀ of cell growth+PARP inhibitor. Growth inhibition curves for both control and compound treated cells are in the presence of the alkylating agent methyl methanesulfonate (MMS). The test compounds were used at a fixed concentration of 0.2 micromolar. The concentrations of MMS were over a range from 0 to 10 μg/ml.

Cell growth was assessed using the sulforhodamine B (SRB) assay (Skehan, P., et al., (1990) New calorimetric cytotoxicity assay for anticancer-drug screening. J. Natl. Cancer Inst. 82, 1107-1112.). 2,000HeLa cells were seeded into each well of a flat-bottomed 96-well microtiter plate in a volume of 100 μl and incubated for 6 hours at 37° C. Cells were either replaced with media alone or with media containing PARP inhibitor at a final concentration of 0.5, 1 or 5 μM. Cells were allowed to grow for a further 1 hour before the addition of MMS at a range of concentrations (typically 0, 1, 2, 3, 5, 7 and 10 μg/ml) to either untreated cells or PARP inhibitor treated cells. Cells treated with PARP inhibitor alone were used to assess the growth inhibition by the PARP inhibitor.

Cells were left for a further 16 hours before replacing the media and allowing the cells to grow for a further 72 hours at 37° C. The media was then removed and the cells fixed with 100 μl of ice cold 10% (w/v) trichloroacetic acid. The plates were incubated at 4° C. for 20 minutes and then washed four times with water. Each well of cells was then stained with 100 μl of 0.4% (w/v) SRB in 1% acetic acid for 20 minutes before washing four times with 1% acetic acid. Plates were then dried for 2 hours at room temperature. The dye from the stained cells was solubilized by the addition of 100 μl of 10 mM Tris Base into each well. Plates were gently shaken and left at room temperature for 30 minutes before measuring the optical density at 564 nM on a Microquant microtiter plate reader.

The following compounds had a PF₅₀ at 500 nM of at least 1.5: 53, 71, 72, 73, 74, 79, 216. Compound 188 had a PF₅₀ at 200 nM of at least 1.5.

1. A compound of the formula (I): 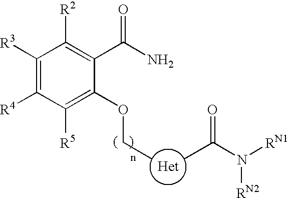
and isomers, salts, solvates, chemically protected forms, and prodrugs thereof, wherein:
R², R³, R⁴ and R⁵ are independently selected from the group consisting of H, C₁₋₇ alkoxy, amino, halo or hydroxy;
n is 1 or 2;
R^{N1} and R^{N2} are independently selected from H and R, where R is optionally substituted C₁₋₁₀ alkyl, C₃₋₂₀ heterocyclyl and C₅₋₂₀ aryl;
or R^{N1} and R^{N2}, together with the nitrogen atom to which they are attached form an optionally substituted 5-7 membered, nitrogen containing, heterocylic ring;
Het is selected from: 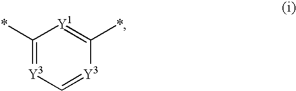
where Y¹ and Y³ are independently selected from CH and N, Y² is selected from CX and N and X is H, Cl or F; and 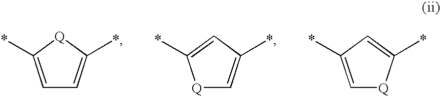
where Q is O or S.

2. A compound according to claim 1, wherein R², R³, R⁴ and R⁵ are selected from the group consisting of H, methoxy, Cl and F.

3. A compound according to claim 1, wherein R², R⁴ and R⁵ are H, and R³ is most selected from H and F.

4. A compound according to claim 1, wherein Het is

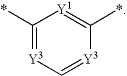

5. A compound according to claim 4, wherein one or none of Y¹, Y² and Y³ are N.

6. A compound according to claim 4, wherein X is selected from H and F.

7. A compound according to claim 1, wherein R^{N1} is H and R^{N2} is R.

8. A compound according to claim 7, wherein R is optionally substituted C₁₋₇ alkyl or C₃₋₂₀ heterocylyl.

9. A compound according to claim 1, wherein R^{N1} and R^{N2}, together with the nitrogen atom to which they are attached form a group of formula II: 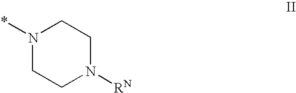
wherein R^{N} is selected from:
(i) -R^{II};
(ii) —C(═O)NHR^{II};
(iii) —C(═S)NHR^{II};
(iv) —S(═O)₂R^{II}; and
(v) —C(═O)R^{II},
where R^{II} is selected from optionally substituted C₁₋₁₀ alkyl, C₃₋₂₀ heterocyclyl and C₅₋₂₀ aryl.

10. A compound according to claim 9, wherein R^{N} is selected from:
(i) —C(═O)NHR^{II};
(ii) —S(═O)₂R^{II}; and
(iii) —C(═O)R^{II}.

11. A compound according to claim 1, wherein R^{N1} and R^{N2}, together with the nitrogen atom to which they are attached form a group of formula III: 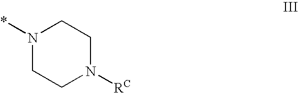
wherein R^{C} is selected from the group consisting of: H; optionally substituted C₁₋₂₀ alkyl; optionally substituted C₅₋₂₀ aryl; optionally substituted C₃₋₂₀ heterocyclyl; optionally substituted acyl; optionally substituted amido; and optionally substituted ester groups.

12. A compound according to claim 11, wherein R^{C} is selected from optionally substituted ester groups.

13. A pharmaceutical composition comprising a compound according to claim 1 and a pharmaceutically acceptable carrier or diluent.

14. A method of treating a disease ameliorated by the inhibition of PARP, comprising administering to a subject in need of treatment a therapeutically-effective amount of a compound according to claim 1.

15. A method of treating cancer, comprising administering to a subject in need of treatment a therapeutically-effective amount of a compound according to claim 1 in combination, simultaneously or sequentially with ionizing radiation or chemotherapeutic agents.

16. A method of treating cancer in an individual, wherein said cancer is deficient in HR dependent DNA DSB repair pathway, comprising administering to a subject in need of treatment a therapeutically-effective amount of a compound according to claim 1.

17. A method according to claim 16, wherein said cancer comprises one or more cancer cells having a reduced or abrogated ability to repair DNA DSB by HR relative to normal cells.

18. A method according to claim 17, wherein said cancer cells have a BRCA1 or BRCA2 deficient phenotype.

19. A method according to claim 18, wherein said cancer cells are deficient in BRCA1 or BRCA2.

20. A method according to claim 16, wherein said treatment further comprises administration of ionising radiation or a chemotherapeutic agent.
